(19) United States
(12) Patent Application Publication
(10) Pub. No.: US 2014/0057868 A1
(43) Pub. Date: Feb. 27, 2014

(54) CATALYTIC HYDROGENOLYSIS OF A COMPOSITION OF A MIXTURE OF OLIGOSACCHARIDE PRECURSORS AND USES THEREOF

(71) Applicants: Gyula Dekany, Sinnamon Park (AU); Sandor Demkó, Debrecen (HU); Andreas Schroven, Barssel (DE); Elise Champion, Toulouse (FR)

(72) Inventors: Gyula Dekany, Sinnamon Park (AU); Sandor Demkó, Debrecen (HU); Andreas Schroven, Barssel (DE); Elise Champion, Toulouse (FR)

(73) Assignee: GLYCOM A/S, Kongens Lyngby (DK)

(21) Appl. No.: 14/000,542

(22) PCT Filed: Feb. 21, 2012
(86) PCT No.: PCT/DK2012/050060

§ 371 Date: Aug. 20, 2013

(30) Foreign Application Priority Data
Feb. 21, 2011 (DK) PA 2011 70092
May 13, 2011 (EP) 11166063.5
May 13, 2011 (EP) 11166135.1
May 13, 2011 (EP) 11166137.7

(51) Int. Cl. A61K 31/702

(52) U.S. Cl. CPC A61K 31/702
USPC 514/61

(57) ABSTRACT

A method for the manufacture of a mixture of human milk oligosaccharides is disclosed. The method involves the catalytic hydrogenolysis of compounds of the general formula 1 and 2. The use of compounds of general formula 1 and 2 in the manufacture of human milk oligosaccharides is also disclosed.

### BACKGROUND OF INVENTION

Human milk oligosaccharides (HMOs) are carbohydrates which have gained much interest in recent years. In particular the synthesis of these HMOs has increased significantly due to the role of HMOs in numerous biological processes occurring in humans. HMOs play a vital role in the early development of young children. Furthermore, the importance of HMOs in the maturation of the immune system and their prognostic use as immunomodulators underlines their importance.

A natural source of such HMOs is mammalian milk. Mammalian milk contains up to 10% HMOs. To date the structure of at least 115 HMOs has been determined while the mass spectra (MS) data has suggested a presence of almost 130 HMOs (Newburg and Neubauer, 1995, Carbohydrates in milks: Analysis, quantities and significance. In: Handbook of Milk Composition (R. G. Jensen, ed.), pp. 273-249, Academic Press, San Diego, USA).

The 115 human milk oligosaccharides, the structures of which have been determined to date, can be grouped into 13 categories based on their core structures. Such 13 categories structures are exemplarily shown in table 1 below (see also Urashima et al., Advanced Dairy Chemistry, Volume 3: Lactose, Water, Salts and Minor Constituents, 2009, pp. 295-349; and TADASU URASHIMA et al, MILK OLIGOSACCHARIDES, Nova Biomedical Books, New York, 2011, ISBN: 978-1-61122-831-1).

TABLE 1
13 different core structures of human milk oligosaccharides (HMOs)

| No | Core name | Core structure |
|---|---|---|
| 1 | lactose (Lac) | Galβ1-4Glc |
| 2 | lacto-N-tetraose (LNT) | Galβ1-3GlcNAcβ1-3Galβ1-4Glc |
| 3 | lacto-N-neotetraose (LNnT) | Galβ1-4GlcNAcβ1-3Galβ1-4Glc |
| 4 | lacto-N-hexaose (LNH) | Galβ1-3GlcNAcβ1-3(Galβ1-4GlcNAcβ1-6)Galβ1-4Glc |
| 5 | lacto-N-neohexaose (LNnH) | Galβ1-4GlcNAcβ1-3(Galβ1-4GlcNAcβ1-6)Galβ1-4Glc |
| 6 | para-lacto-N-hexaose (para-LNH) | Galβ1-3GlcNAcβ1-3Galβ1-4GlcNAcβ1-3Galβ1-4Glc |
| 7 | para-lacto-N-neohexaose (para-LNnH) | Galβ1-4GlcNAcβ1-3Galβ1-4GlcNAcβ1-3Galβ1-4Glc |
| 8 | lacto-N-octaose (LNO) | Galβ1-3GlcNAcβ1-3(Galβ1-4GlcNAcβ1-3Galβ1- |
| | | 4GlcNAcβ1-6)Galβ1-4Glc |
| 9 | lacto-N-neooctaose (LNnO) | Galβ1-4GlcNAcβ1-3(Galβ1-3GlcNAcβ1-3Galβ1- |
| | | 4GlcNAcβ1-6)Galβ1-4Glc |
| 10 | Iso-lacto-N-octaose (iso-LNO) | Galβ1-3GlcNAcβ1-3(Galβ1-3GlcNAcβ1-3Galβ1- |
| | | 4GlcNAcβ1-6)Galβ1-4Glc |
| 11 | para-lacto-N-octaose (para-LNO) | Galβ1-3GlcNAcβ1-3Galβ1-4GlcNAcβ1-3Galβ1- |
| | | 4GlcNAcβ1-3Galβ1-4Glc |
| 12 | Lacto-N-neodecaose (LNnD) | Galβ1-3GlcNAcβ1-3[Galβ1-4GlcNAcβ1-3(Galβ1- |
| | | 4GlcNAcβ1-6)Galβ1-4GlcNAcβ1-6]Galβ1-4Glc |
| 13 | Lacto-N-decaose (LND) | Galβ1-3GlcNAcβ1-3[Galβ1-3GlcNAcβ1-3(Galβ1- |
| | | 4GlcNAcβ1-6)Galβ1-4GlcNAcβ1-6]Galβ1-4Glc |

Due to the large number of HMOs and their low concentrations in mammalian milk, an isolation of HMOs from mammalian milk is a difficult task. It is therefore difficult to provide suitable HMOs replacements in foods, particularly in infant formulae which display at least part of the entire spectrum of HMOs.

Although methods for the manufacture of HMOs are known, be it chemically or enzymatically, such manufacturing methods do not allow the preparation of mixtures of HMOs. Preparing such mixtures of HMOs on the basis of individually designed syntheses of single HMOs is furthermore costly and may not resemble the large variety of naturally occurring HMOs.

There is a need to provide a means for the manufacture of a mixture of HMOs wherein the mixture of HMOS has a profile which resembles a mixture of HMOs as found in human milk.

There is also a need to be able to provide a method for the manufacture of HMOs on a relatively large scale, which avoids the use of complicated and expensive methods such as those methods which utilises biotechnology.

There is a need to provide a method, which allows for the manufacture of a mixture of HMOs on a large scale.

### SUMMARY OF INVENTION

In a first aspect the present invention to a composition comprising a mixture of at least two compounds selected from compounds of general formulae 1 and 2

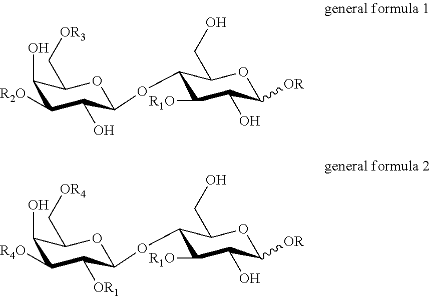

wherein R is a group removable by catalytic hydrogenolysis,
R₁ is independently fucosyl or H,
R₂ is selected from N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₃ is H or N-acetyl-lactosaminyl group optionally substituted with a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₄ is independently sialyl or H,
and salts thereof,
provided that at least one of R₁ or R₄ is not H in general formula 2, and
the compound is not 3′-sialyllactose benzyl glycoside sodium salt, 6′-sialyllactose benzyl glycoside sodium salt, LNT benzyl glycoside, and LNnT benzyl glycoside.

In a further aspect a use of the composition of general formulae 1 and 2 in the manufacture of a consumable product is disclosed.

In a further aspect a method for the manufacture of a mixture of human milk oligosaccharides (HMOs) is disclosed. The method comprises subjecting a mixture of at least two compounds selected from compounds of general formulae 1 and 2 to catalytic hydrogenolysis to remove the R group.

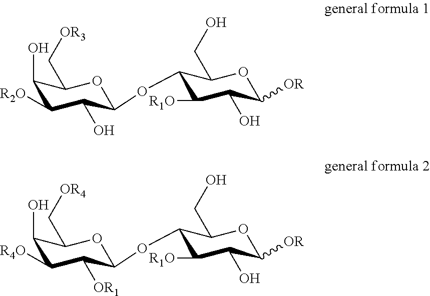

R is a group removable by catalytic hydrogenolysis,
R₁ is independently fucosyl or H,
R₂ is selected from N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₃ is H or N-acetyl-lactosaminyl group optionally substituted with a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₄ is independently sialyl or H,
and salts thereof,
with the proviso that at least one of R₁ or R₄ is not H in general formula 2.

### DETAILED DESCRIPTION OF THE INVENTION

For a complete understanding of the present invention and the advantages thereof, reference is made to the following detailed description.

It should be appreciated that various embodiments of the present invention can be combined with other embodiments of the invention and are merely illustrative of the specific ways to make and use the invention and do not limit the scope of the invention when taken into consideration with the claims and the following detailed description.

### The Composition of the Invention

Preferably, the object underlying the present invention is solved by providing a composition comprising a mixture of at least two compounds selected from compounds of general formulae 1 and 2

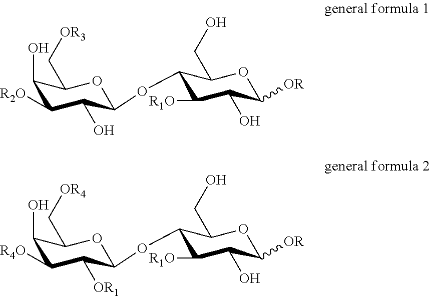

wherein R is a group removable by catalytic hydrogenolysis,
R₁ is independently fucosyl or H,
R₂ is selected from N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₃ is H or N-acetyl-lactosaminyl group optionally substituted with a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₄ is independently sialyl or H,
and salts thereof,
with the proviso that at least one of R₁ or R₄ is not H in general formula 2.

A mixture of HMOs is manufactured by catalytic hydrogenolysis of compounds of general formulae 1 and 2.

In the context of the present invention the expression “group removable by catalytic hydrogenolysis” refers to groups, whose C-0 bond is cleaved by addition of hydrogen in the presence of a hydrogenolysis catalyst. The hydrogenolysis catalyst is used in the presence of hydrogen gas under pressure and with heat. The hydrogenolysis catalyst can be for example palladium, Raney nickel, palladium on charcoal or palladium black or another appropriate metal catalyst known for use in hydrogenolysis. Thus the use of the hydrogenolysis catalyst in the present invention results in the regeneration of —OH group from the R groups of compounds of general formulae 1 and 2. The R groups of this type are known (see e.g. P. G. M. Wuts and T. W. Greene: Protective Groups in Organic Synthesis, John Wiley & Sons (2007)). Suitable R groups include benzyl, diphenylmethyl (benzhydryl), 1-naphthylmethyl, 2-naphthylmethyl or triphenylmethyl (trityl) groups, each of which may be optionally substituted by one or more groups selected from: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substitution, if present, is on the aromatic ring(s). Particularly preferred protecting group is benzyl optionally substituted with one or more groups selected from alkyl or halogen. More preferably, the protecting group is selected from unsubstituted benzyl, 4-chlorobenzyl and 4-methylbenzyl. These particularly preferred and more preferable protecting groups have the advantage that the by-products of the hydrogenolysis are exclusively toluene or substituted toluene. Such by-products can easily be removed even in multi ton scales from water soluble oligosaccharide products via evaporation and/or extraction processes.

Additionally, the term “fucosyl” within the context of the present invention preferably means a L-fucopyranosyl group attached to the oligosaccharide of compounds of general formulae 1 and 2 by an α-interglycosidic linkage, such that:

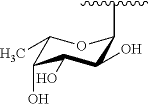

“N-acetyl-lactosaminyl” group within the context of the present invention preferably means the glycosyl residue of N-acetyl-lactosamine (LacNAc, Galpβ1-4GlcNAcp) of compounds of general formulae 1 and 2 links with β-linkage such that:

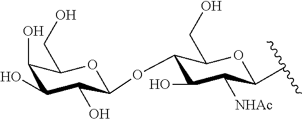

Furthermore, the term “Lacto-N-biosyl” group within the context of the present invention preferably means the glycosyl residue of lacto-N-biose (LNB, Galpβ1-3GlcNAcp) of compounds of general formulae 1 and 2 links with β-linkage such that:

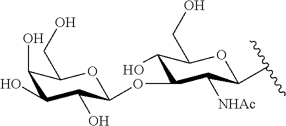

The term “Sialyl” within the context of the present invention preferably means the glycosyl residue of sialic acid (N-acetyl-neuraminic acid, Neu5Ac)) of compounds of general formulae 1 and 2 links with β-linkage such that:

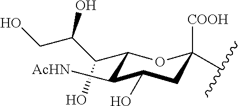

The term “glycosyl residue comprising one or more N-acetyl-lactosaminyl and/or one or more lacto-N-biosyl units” within the context of the present invention preferably means a linear or branched structure comprising the said units that are linked to each other by interglycosidic linkages.

### The Method of the Invention

According to the method mentioned above, at least two least two compounds selected from compounds of general formulae 1 and 2 wherein R is a group removable by catalytic hydrogenolysis are provided. Such a mixture of compounds is preferably as noted is at least two, however three, four, five, two to five, five to ten, two to ten, two to twenty, three to twenty, four or even five to twenty, or even more of the compounds selected from compounds of general formulae 1 and 2.

The claimed method is based upon the utilisation of at least two compounds selected from compounds characterized by general formulae 1 and 2 defined above in catalytic hydrogenolysis. This reaction typically takes place in a protic solvent or in a mixture of protic solvents. A protic solvent may be selected from water, acetic acid or C₁-C₆ alcohols. A mixture of one or more protic solvents with one or more suitable aprotic organic solvents partially or fully miscible with the protic solvent(s) (such as THF, dioxane, ethyl acetate or acetone) may also be used. Water, one or more C₁-C₆, alcohols or a mixture of water and one or more C₁-C₆, alcohols are preferably used as the solvent system. Solutions containing the carbohydrate derivatives in any concentration or suspensions of the carbohydrate derivatives in the solvent(s) used are also applicable. The reaction mixture is stirred at a temperature in the range of 10-100° C., preferably between 20-50° C., in a hydrogen atmosphere of 1-50 bar absolute (100 to 5000 kPa) in the presence of a catalyst such as palladium, Raney nickel or any other appropriate metal catalyst, preferably palladium on charcoal or palladium black, until reaching the completion of the reaction. Transfer hydrogenolysis may also be performed, when the hydrogen is generated in situ from cyclohexene, cyclohexadiene, formic acid or ammonium formate. Addition of organic or inorganic bases or acids and/or basic and/or acidic ion exchange resins can also be used to improve the kinetics of the hydrogenolysis. The use of basic substances is especially preferred when halogen substituents are present on the substituted benzyl moieties of the precursors and/or the formation of mannosamine base is desirable. Preferred organic bases include, but are not limited to, triethylamine, diisopropyl ethylamine, ammonia, ammonium carbamate and diethylamine. An organic or an inorganic acid is favourably used as a co-solvent or additive in cases when mannosamine salts are the intended products. Preferred acids include, but are not limited to, formic acid, acetic acid, propionic acid, chloroacetic acid, dichloroacetic acid, trifluoroacetic acid, HCl and HBr. The conditions proposed above allow simple, convenient and delicate removal of the solvent(s) giving rise to pure HMO mixture or blend.

In a preferred embodiment at least two compounds selected from compounds characterized by general formulae 1 and 2 defined above are subjected to catalytic hydrogenolysis to provide at least two HMOs. The catalytic hydrogenolysis can be performed in water or in aqueous alcohol, preferably in water, water/methanol or water/ethanol mixture (alcohol content: 10-50 v/v %). The catalytic hydrogenolysis is performed at a temperature of between 15-65° C., preferably between 40-60° C. The catalyst concentration may range from 0.4% to 1.2% (weight of the metal content based on the weight of the starting carbohydrate mixture).

The compounds of general formulae 1 are defined under general formulae 1a, 1b. The compounds of general formulae 2 are defined under general formulae 2 below.

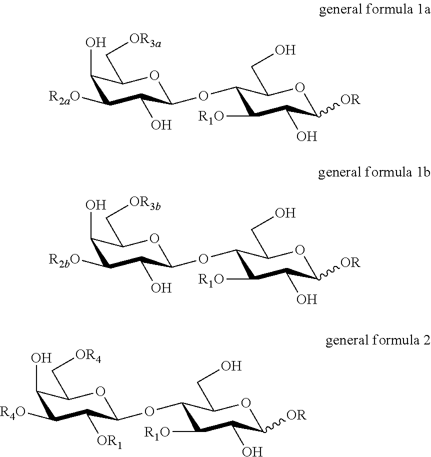

wherein R, R₁ and R₄ are as defined above,
R₂ₐ is N-acetyl-lactosaminyl group optionally substituted with a glycosyl residue comprising one N-acetyl-lactosaminyl and/or one lacto-N-biosyl group; any N-acetyl-lactosaminyl and lacto-N-biosyl group can be substituted with one or more sialyl and/or fucosyl residue,
R₃ₐ is H or N-acetyl-lactosaminyl group optionally substituted with a lacto-N-biosyl group; any N-acetyl-lactosaminyl and lacto-N-biosyl group can be substituted with one or more sialyl and/or fucosyl residue,
R_{2b} is lacto-N-biosyl group optionally substituted with sialyl and/or fucosyl residue,
R_{3b} is H or N-acetyl-lactosaminyl group optionally substituted with one or two N-acetyl-lactosaminyl and/or one lacto-N-biosyl group; any N-acetyl-lactosaminyl and lacto-N-biosyl group can be substituted with one or more sialyl and/or fucosyl residue,
are provided for hydrogenolysis.

It is preferable that
the N-acetyl-lactosaminyl group in the glycosyl residue of R₂ₐ in general formula is attached to the another N-acetyl-lactosaminyl group with 1-3 interglycosidic linkage,
the lacto-N-biosyl group in the glycosyl residue of R₂ₐ in general formula is attached to the N-acetyl-lactosaminyl group with 1-3 interglycosidic linkage,
the lacto-N-biosyl group in the glycosyl residue of R₃ₐ in general formula is attached to the N-acetyl-lactosaminyl group with 1-3 interglycosidic linkage,
the N-acetyl-lactosaminyl group in the glycosyl residue of R_{3b} in general formula 1b is attached to the another N-acetyl-lactosaminyl group with 1-3 or 1-6 interglycosidic linkage,
the lacto-N-biosyl group in the glycosyl residue of R_{3b} in general formula 1b is attached to the N-acetyl-lactosaminyl group with 1-3 interglycosidic linkage.

It is further preferable that compounds of general formula 1a and 1b, wherein general formula is represents the R-glycosides of lacto-N-neotetraose, para-lacto-N-hexaose, para-lacto-N-neohexaose, lacto-N-neohexaose, para-lacto-N-octaose and lacto-N-neooctaose optionally substituted with one or more sialyl and/or fucosyl residue, and general formula 1b represents the R-glycosides of lacto-N-tetraose, lacto-N-hexaose, lacto-N-octaose, iso-lacto-N-octaose, lacto-N-decaose and lacto-N-neodecaose optionally substituted with one or more sialyl and/or fucosyl residue.

Particularly preferable compounds used for hydrogenolysis of general formula 1 and 2 are wherein:
the fucosyl residue attached to the N-acetyl-lactosaminyl and/or the lacto-N-biosyl group is linked to
the galactose of the lacto-N-biosyl group with 1-2 interglycosidic linkage and/or
the N-acetyl-glucosamine of the lacto-N-biosyl group with 1-4 interglycosidic linkage and/or
the N-acetyl-glucosamine of the N-acetyl-lactosaminyl group with 1-3 interglycosidic linkage,
the sialyl residue attached to the N-acetyl-lactosaminyl and/or the lacto-N-biosyl group is linked to
the galactose of the lacto-N-biosyl group with 2-3 interglycosidic linkage and/or
the N-acetyl-glucosamine of the lacto-N-biosyl group with 2-6 interglycosidic linkage and/or
the galactose of the N-acetyl-lactosaminyl group with 2-6 interglycosidic linkage.

The most preferable R-glycosides provided for hydrogenolysis represent naturally occurring HMOs having a lactose, LNT or LNnT core, and are selected from: R-glycosides of 2′-fucosyllactose, 3-fucosyllactose, 2′,3-difucosyllactose, 3′-sialyllactose, 6′-sialyllactose, 3′-sialyl-3-fucosyllactose, lacto-N-tetraose, lacto-N-neotetraose, LNFP-I, LNFP-II, LNFP-III, LNFP-V, LST-a, LST-b, LST-c, FLST-a, FLST-b, FLST-c, LNDFH-I, LNDFH-II, LNDFH-III, DS-LNT, FDS-LNT I and FDS-LNT II.

According to another preferred embodiment, the compounds used in hydrogenolysis specified above are β-glycosides, more preferably the aglycon is benzyl.

### Preparation of the Compounds of General Formulae 1 and 2

The individual compounds comprised in the mixture or blend ready for hydrogenolysis can be synthesized by chemical, enzymatic or chemo-enzymatic way. For example the production of 1-O-benzyl/substituted benzyl-LNnT is described in Ponpipom et al. Tetrahedron Lett. 20, 1717 (1978) and in international application WO 2011/100980; 1-O-benzyl/substituted benzyl-6′-SL and their salt are disclosed in international application WO 2011/100979; the production of 1-O-benzyl-LNT is published in Liu et al. Bioorg. Med. Chem. 17, 4910 (2009); 1-O-benzyl-3′-SL sodium salt is specified in international application WO 96/32492 A1.

According to another method to make mixture or blend of compounds of general formulae 1 and 2, the synthesis of a mixture of sialylated human milk precursors comprises coupling a protected sialyl donor, such as protected sialyl halide, thioglycoside, trichloroacteimidate, phosphate, phosphite, etc. with a mixture comprising two or more desialo-human milk oligosaccharide R-glycosides in protected form. Similarly, the synthesis of a mixture of fucosylated human milk comprises coupling a protected fucosyl donor, such as protected fucosyl halide, thioglycoside, trichloroacteimidate, phosphate, phosphite, etc. with a mixture comprising two or more defuco-human milk oligosaccharide R-glycosides in protected form. The mixture of coupled products so obtained is in fact a mixture of protected sialylated human milk oligosaccharides or a mixture of protected fucosylated human milk oligosaccharides. They can be subjected to remove the protective groups present. Removal of the masking groups can be carried out in one step or more consecutive steps. It is within the skilled person competence to select the appropriate reagent(s) and condition(s) for this purpose. The mixture of sialylated human milk oligosaccharide R-glycosides or the mixture of protected fucosylated human milk oligosaccharide R-glycosides can be isolated from the reaction mixture using conventional work-up procedures both in solid form such as amorphous/freeze dried/spray dried or crystalline form and in liquid form as syrup or concentrated aqueous solution, and they are ready for hydrogenolysis.

### Preparation Using Phosphite Donors

Alternatively, the individual compounds comprised in the mixture, or even mixtures of compounds may be prepared using phosphite donors of general formula I

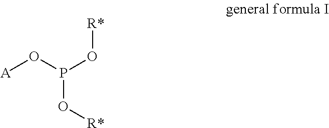

wherein A is glycosyl residue of a mono-, di- or oligosaccharide in protected form and R* is selected from optionally substituted aryl or optionally substituted heteroaryl.

When R* denotes optionally substituted heteroaryl, it is attached to the oxygen atom by a carbon atom of the aromatic ring.

The term “glycosyl residue of a mono-, di- or oligosaccharide in protected form” intends to mean any derivatized or non-derivatized mono-, di- or oligosaccharide glycosyl residue which is attached to the —O—P(OR*)₂ phosphityl group by the C-1 (aldoses) or C-2 (ketoses) anomeric carbon atom thus forming glycosyl phosphite type compounds. If the glycosyl residue differs from monosaccharide, it may represent a linear or branched structure consisting of monosaccharide units that are linked to each other by interglycosidic linkages. The monosaccharide or monosaccharides units can be selected from any 5-9 carbon atom containing sugars consisting of aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglycosamine, N-acetylmannosamine, N-acetylgalactosamine, etc.), uronic acids, ketoaldonic acids (e.g. sialic acid) and like. The functional groups of the glycosyl residue are protected and/or derivatized. The protective groups can be the commonly used ones in organic/carbohydrate chemistry; they are well known to the skilled man and are discussed e.g. in P. G. M. Wuts and T. W. Greene: Protective Groups in Organic Synthesis, John Wiley & Sons (2007); S. Hanessian: Preparative Carbohydrate Chemistry Marcel Dekker (1997); Chemical Synthesis of Glycosides and Glycomimetics in: Carbohydrates in Chemistry and Biology (Eds.: B. Ernst, G. W. Hart, P. Sinaÿ) Part I, Vol. 1, Wiley (2000).

According to a preferred embodiment of compounds of general formula I, A means a sialyl moiety in protected form or a fucosyl moiety in protected form, such as a sialyl moiety in protected form, and R* is optionally substituted aryl.

The term “sialyl moiety in protected form” refers to glycosyl residue of any naturally occurring or modified neuraminic or sialic acid derivatives and analogues thereof in protected form. In a sialyl moiety the C-2 (anomeric) carbon atom is attached to the phosphityl residue. Preferred neuraminic acids are N-acetyl-(Neu5Ac), N-glycolyl-(Neu5Gc) and deamino-neuraminic acid (3-deoxy-D-glycero-D-galacto-nonulosonic acid, KDN). Also included are Neu5Ac, Neu5Gc and KDN derivatives that are derivatized with linkers, reactive functional groups, detectable labels or targeting moieties. The derivatization may affect C-3 with the introduction of bulky thio groups, C-4 with the introduction of piperidino, piperazino or morpholino moieties and C-5 with the introduction of azido group and formation of 5-N,4-O-cyclic carbamate. The protective groups on the sialyl moiety are to mask hydroxyls (as ethers and/or esters and/or acetals), —NHAc or —NH₂ (as amides or carbamates) and the carboxylic portion (as esters or thioesters) and commonly used in organic/carbohydrate chemistry. Thus C-5 nitrogen can be protected e.g. as N,N-diacetyl, N-trifluoroacetyl, N-trichloroacetyl, N-phthalyl, N-Troc, N-Fmoc and the like.

The term “fucosyl moiety in protected form” refers to a fucopyranosyl moiety attached to the phosphityl residue via the anomeric carbon atom, and in which the hydroxyl groups are protected as ethers and/or esters and/or acetals or by other means commonly used in organic/carbohydrate chemistry.

A more preferred embodiment encompasses compounds of general formulae IIA and IIB, such as compounds of general formula IIA,

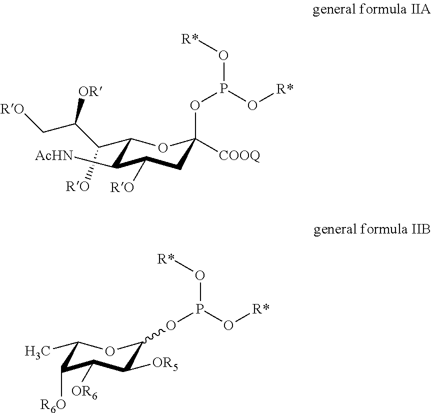

wherein R* is optionally substituted phenyl, R′ is optionally substituted acyl, Q is optionally substituted alkyl, R₅ is a group removable by hydrogenolysis or optionally substituted acyl, and R₂ is a group removable by hydrogenolysis, optionally substituted acyl or two R₆ groups together form a moiety

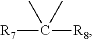

wherein R₇ and R₈, independently, are alkyl or phenyl, or wherein groups R₇ and R₈ together with the carbon atom to which they are attached form cycloalkylidene.

An especially preferred embodiment relates to compounds of general formulae IIIA and IIIB, such as compounds of general formula IIIA,

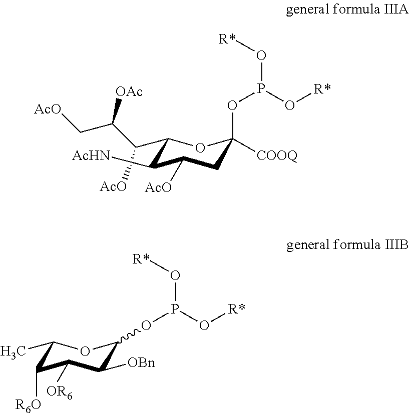

wherein Q is selected from C₁₋₆ alkyl and benzyl, preferably methyl, R* is phenyl optionally substituted with alkyl, alkoxy and/or halogen, preferably methyl, methoxy and/or bromo, and R₆ is benzyl, acetyl or benzoyl optionally substituted with chloro.

Unlike to other glycosyl phosphites specified in the art, compounds of general formula I, preferably of general formulae IIA and IIB, even more preferably of general formulae IIIA and IIIB, such as of general formula IIIA, of the present application can be considered as crystalline materials. They are stable, can be stored for longer period of time without significant decomposition, can be easily activated in glycosylation reactions and show excellent α-selectivity. Accordingly, compounds of general formulae IIIA and IIIB, such as of general formula IIIA, have obviously advantageous applicability in sialylation or fucosylation reactions, such as in sialylation reactions.

The phosphite donors of formula I can be prepared according to a method wherein a compound of formula A-OH, wherein A means glycosyl residue of a mono-, di- or oligosaccharide in protected form is reacted with
a) a compound of (R*O)₂PY in which R* is selected from optionally substituted aryl and optionally substituted heteroaryl and Y is selected from halogen and dialkylamino, or
b) PX₃ wherein X is halogen, followed by reaction with an alcohol R*OH wherein R* is defined as above.

In a compound of formula A-OH A is glycosyl residue of a mono-, di- or oligosaccharide in protected form as defined above, thus compound of A-OH represents any derivatized or non-derivatized protected mono-, di- or oligosaccharide in protected form with free glycosidic OH.

The reactions are typically carried out in aprotic solvent or mixture aprotic solvents, such as halogenated solvents like dichloromethane or chloroform, DMF, dioxane, toluene, acetonitrile, etc. When conducting the synthesis according to option a) tertiary amines like triethyl amine or Hünig's base are employed to scavenge the liberating acid HY. As to option b) in the first step N-containing aromatic bases like pyridine, imidazole, tetrazole are the preferred base of choice then in the subsequent condensation step tertiary amines like triethyl amine or Hünig's base are used to neutralize acids. The two types of base can be added to the reaction mixture in the same time or consecutively.

In a preferred method option b) is taken wherein the first reaction is carried out at 0-25° C., preferably at 5-10° C., then the second reaction is conducted at 20-40° C., preferably at room temperature. As to reactants and reagents, the preferred anomerically free sugar is a suitably protected sialic acid derivative, more preferably N-acetyl neuraminic acid tetraacetate methyl ester, PX₃ is PCl₃ and the aromatic alcohol R*OH is phenol optionally substituted with alkyl, alkoxy and/or halogen, preferably methyl, methoxy and/or bromo.

In another preferred method option b) is taken wherein the first reaction is carried out at 0-25° C., preferably at 5-10° C., then the second reaction is conducted at 20-40° C., preferably at room temperature. As to reactants and reagents, the preferred anomerically free sugar is a suitably protected fucose derivative, preferably 2-O-benzyl-3,4-di-O-(optionally substituted acyl)-L-fucose, PX₃ is PCl₃ and the aromatic alcohol R*OH is phenol optionally substituted with alkyl, alkoxy and/or halogen, preferably methyl, methoxy and/or bromo.

In connection with the phosphite donors, the term “alkyl”, either alone or when attached to another atom or group, means a linear or branched hydrocarbon group with 1-20 carbon atoms, preferably with 1-6 carbon atoms, like methyl, ethyl, n-propyl, i-propyl, n-butyl, i-butyl, s-butyl, t-butyl, etc. The term “cycloalkylidene” means a bivalent cyclic hydrocarbon ring having 3-8 carbon atoms, such as cyclopropylidene, cyclopentylidene, cyclohexylidene, cycloheptylidene, etc. The term “aryl” refers to homoaromatic groups like phenyl or naphthyl. Preferably, aryl means phenyl. The term “heteroaryl” refers to aromatic groups having one or two rings, which ring(s) contain(s) 1, 2, or 3 heteroatoms selected from N, O and S, such as pyrrol, imidazole, pyrazole, 1,2,3-triazole, 1,2,4-triazole, furan, thiophene, oxazole, isoxazole, thiazole, thiadiazole, pyridine, pyrazine, pyridazine, pyrimidine, triazine, benzimidazole, benzoxazole, benzthiazole, indole, quinoline, isoquinoline, purine, pteridine and the like. The term “acyl” represent an R″—C(═O)—, wherein R″ may be H, alkyl or aryl, like formyl, acetyl, propionyl, butyryl, pivaloyl, benzoyl, etc. The alkyl and aryl residues both may be substituted. The term “group removable by hydrogenolysis” means a protecting group whose C—O bond to the oxygen can be cleaved by hydrogen in the presence of a catalytic amount of palladium, Raney nickel or any other conventional hydrogenolysis catalyst to regenerate the OH group. Such protecting groups are described in Wuts and Greene: Protective Groups in Organic Synthesis, John Wiley & Sons, 2007, and include benzyl, diphenylmethyl (benzhydryl), 1-naphthylmethyl, 2-naphthylmethyl and triphenylmethyl (trityl) groups, each of which can be optionally substituted by one or more of the following groups: alkyl, alkoxy, phenyl, amino, acylamino, alkylamino, dialkylamino, nitro, carboxyl, alkoxycarbonyl, carbamoyl, N-alkylcarbamoyl, N,N-dialkylcarbamoyl, azido, halogenalkyl or halogen. Preferably, such substitution, if present, is on the aromatic ring(s). A preferred protecting group is benzyl optionally substituted with one or more of the following groups: phenyl, alkyl and halogen, particularly unsubstituted benzyl, 4-chlorobenzyl, 3-phenylbenzyl and 4-methylbenzyl groups. The term “optionally substituted” means that the group in question may either carry a substituent or may be unsubstituted. The term “substituted” means that the group in question is substituted with a group which modifies the general chemical characteristics of the chain or ring. The substituents can be used to modify characteristics of the molecule as a whole, such as stability, solubility, and ability to form crystals. The person skilled in the art will be aware of other suitable substituents of a similar size and charge characteristics, which could be used as alternatives in a given situation. More generally in connection with the terms “alkyl”, “cycloalkylidene”, “aryl”, “heteroaryl” and “acyl” the term “optionally substituted” is intended to mean that the group in question may be substituted one or several times, preferably 1-5 times, more preferably 1-3 times with group(s) selected from alkyl (only for aryl, heteroaryl and aromatic acyl), hydroxy, alkoxy (i.e. alkyl-oxy), carboxy, oxo (forming a keto or aldehyde functionality), alkoxycarbonyl, alkylcarbonyl, formyl, aryl, aryloxycarbonyl, aryloxy, arylamino, arylcarbonyl, amino, mono- and dialkylamino, carbamoyl, mono- and dialkyl-aminocarbonyl, alkylcarbonylamino, cyano, alkanoyloxy, nitro, alkylthio and halogen (F, Cl, Br, I).

Compounds of general formulae I, IIA, IIB, IIIA and IIIB can be readily used in glycosidation, preferably sialylation and fucosylation reactions, such as sialylation reactions. Promoters can be selected, as in case of other glycosyl phosphites, from the group of Lewis acids like TMSOTf, BF₃.OEt₂, NIS, TfOH, LiClO₄, iodine, montmorillonite, Tf₂NH, ZnCl₂, Tf₂O or mixture thereof. The reaction runs in aprotic solvent, preferably in dichloromethane, THF, toluene, acetonitrile or in mixtures thereof, more preferably in dichloromethane/THF mixture, at temperatures between −78-0° C., preferably between −15 and −25° C.

Synthesis of oligosaccharides including sialooligosaccharides and fucooligosaccharides, such as sialooligosaccharides, generally follows multistep reaction sequence consisting of orthogonal protection—glycosylation—selective deprotection cascades until the target is reached. In large scale or industrial realization adjusting technological time is among crucial concerns. The main drawback of the multistep procedures is the unavoidable chromatographic separation in order either to get the pure substance/intermediate or to obtain at least a mixture that is enriched in the target compound but still contains undesired derivatives. Although repeated chromatographic separation may results in the improvement of the purity, its high cost and relatively long technological time to handle the feed solution and the column packing, to carry out the separation and optionally to regenerate the packing, especially in large or industrial scale, can be disadvantageous and/or cumbersome.

Crystallization or recrystallization is one of the simplest and cheapest methods to isolate a product from a reaction mixture, separate it from contaminations and obtain pure substance. Isolation or purification that uses crystallization makes the whole technological process robust and cost-effective, thus it is advantageous and attractive compared to other procedures.

Compounds of general formulae I, IIA, IIB, IIIA and/or IIIB excellent choice as general/sialyl/fucosyl donor for glycosylation/sialylation/fucosylation reaction. As they can be synthesized in simple way in crystalline form with high purity and possess remarkable shelf-life they have obviously advantageous usability compared to other phosphites.

Accordingly, an oligosaccharide, in particular compounds of general formulae 1 and 2, can be prepared by a synthesis comprising at least the step of: coupling a compound of general formula I as defined above with an acceptor of the formula B—OH, wherein B—OH means a mono-, di- or oligosaccharide in protected form.

The acceptor B—OH in suitably protected form means any derivatized or non-derivatized mono-, di- or oligosaccharide whose functional groups are protected except for the OH-group to be coupled. In exceptional cases the group B may contain 1 or 2 additional free hydroxyl groups, preferably whose reactivity is much diminished than of that to be coupled, e.g. due to steric hindrance. The unprotected OH-group or one of the OH-groups is preferably not anomeric OH. The protective groups on compound B—OH may be identical, similar or different to those present in the donor of general formula I. If the glycosyl residue in group B differs from monosaccharide, it may represent a linear or branched structure, consisting of monosaccharide units that are attached to each other by interglycosidic linkages. The monosaccharides units in compounds B—OH can be selected from any 5-9 carbon atom containing sugars consisting of aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglycosamine, N-acetylmannosamine, N-acetylgalactosamine, etc.), uronic acids, ketoaldonic acids (e.g. sialic acid) and like. The protective groups can be the commonly used ones in organic/carbohydrate chemistry, such groupings have been mentioned above.

The coupling reaction can be carried out in the presence of promoters can be selected, as in case of other glycosyl phosphites, from the group of Lewis acids like TMSOTf, BF₃.OEt₂, NIS, TfOH, LiClO₄, iodine, montmorillonite, Tf₂NH, ZnCl₂, Tf₂O or mixture thereof. The reaction runs in aprotic solvent, preferably in dichloromethane, THF, toluene, acetonitrile or in mixtures thereof, more preferably in dichloromethane/THF mixture, at temperatures between −78-0° C., preferably between −15 and −25° C.

The coupled product of formula A-O—B can thus be obtained which is a protected di- or oligosaccharide. If di- or oligosaccharide of formula A-O—B has been targeted to synthesize, the compounds is then subjected to remove the protective groups present. Removal of the masking groups can be carried out in one step or more consecutive steps. It is within the skilled person competence to select the appropriate reagent(s) and condition(s) for this purpose. For general considerations it is referred to the following books and reviews: P. G. M. Wuts and T. W. Greene: Protective Groups in Organic Synthesis, John Wiley & Sons (2007); S. Hanessian: Preparative Carbohydrate Chemistry Marcel Dekker (1997); Chemical Synthesis of Glycosides and Glycomimetics in: Carbohydrates in Chemistry and Biology (Eds.: B. Ernst, G. W. Hart, P. Sinaÿ) Part I, Vol. 1, Wiley (2000).

In case of need, compounds of formula A-O—B can be deprotected selectively to set free OH-group(s) for further manipulations such as glycosylation or derivatization. The skilled person is capable of choosing the appropriate reagent(s) and condition(s) in order to deprotect one or more functional groups while the other groups remain intact. In a certain phase of the reaction sequence the so obtained oligosaccharide can then be deprotected (vide supra).

In preferred embodiment compounds of general formula I are in fact compounds of general formulae IIA or IIB, such as compounds of general formula IIA, as defined above. In this particular case a compound of formula B—OH is sialylated or fucosylated giving rise to a compound of general formulae IVA or IVB, such as of general formula IVA,

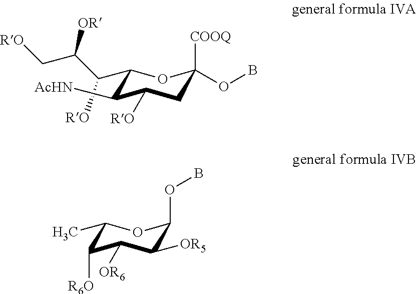

wherein R′ is optionally substituted acyl, Q is optionally substituted alkyl, R₅ is a group removable by hydrogenolysis or optionally substituted acyl, and R₆ is a group removable by hydrogenolysis, optionally substituted acyl or two R₆ groups together form a moiety

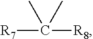

wherein R, and Rₛ, independently, are alkyl or phenyl, or wherein groups R, and R₈ together with the carbon atom to which they are attached form cycloalkylidene, and B is a mono-, di- or oligosaccharide in suitably protected form as defined above. Compounds of general formulae IVA and IVB are protected/partially protected sialo- or fucooligosaccharides with α-glycosidic linkage.

A more preferred embodiment relates to the synthesis of compounds of general formulae VA and VB. such as of general formula VA,

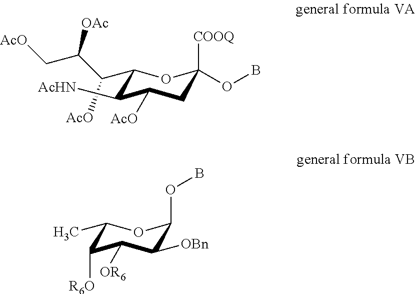

wherein Q is C₁₋₆ alkyl or benzyl, preferably methyl, R₆ is benzyl, acetyl or benzoyl optionally substituted with chloro, and B is a mono-, di- or oligosaccharide in protected form as defined above, using compounds of general formulae IIIA and IIIB, such as of general formula IIIA, as defined above.

An even more preferred embodiment relates to the synthesis of a sialylated or fucosylated human milk oligosaccharide, such as a sialylated human milk oligosaccharide, characterized in that the said synthesis comprises at least the step of: coupling a compound of general formulae IIIA or IIIB, such as of general formula IIIA, with an acceptor of the formula C—OH, wherein C—OH means a desialo- or defuco-human milk oligosaccharide in protected form.

Among sialo- and fucoglycoconjugates, sialylated or fucosylated human milk oligosaccharides like 3′-sialyllactose, 6′-sialyllactose, 2′-fucosyllactose, 3-fucosyllactose, 3′-sialyl-3-fucosyllactose, 2′,3-difucosyllactose, sialyllacto-N-tetraoses (LST a, LST b, LST c), sialyl-fucosyllacto-N-tetraoses (FLST a, FLST b, FLST c), lacto-N-fucopentaoses (LNFP I, LNFP II, LNFP III, LNFP V), lacto-N-difucohexaoses (LNDFH I, LNDFH II, LNDFH III), disialyllacto-N-tetraoses, sialyllacto-N-fucopentaoses, monosialyllacto-N-hexaoses, monosialyllacto-N-neohexaoses, monofucosyllacto-N-hexaoses, monofucosyllacto-N-neohexaoses, monofucosyl-monosialyllacto-N-hexaoses, monofucosyl-monosialyllacto-N-neohexaoses, monofucosyl-disialyllacto-N-hexaoses, monofucosyl-disialyllacto-N-neohexaoses, etc., such as 3′-sialyllactose, 6′-sialyllactose, 3′-sialyl-3-fucosyllactose, sialyllacto-N-tetraoses, sialyl-fucosyllacto-N-tetraoses, disialyllacto-N-tetraose, sialyllacto-N-fucopentaose, monosialyllacto-N-hexaose, monofucosyl-monosialyllacto-N-hexaose, monofucosyl-monosialyllacto-N-neohexaose, monofucosyl-disialyllacto-N-neohexaose, etc., are of great importance which is directly linked to their unique biological activities such as antibacterial, antiviral, immune system and cognitive development enhancing activities. Sialylated and fucosylated human milk oligosaccharides, such as sialylated human milk oligosaccharides, are found to act as prebiotics in the human intestinal system helping to develop and maintain the intestinal flora. Furthermore they have also proved to be anti-inflammatory, and therefore these compounds are attractive components in the nutritional industry for the production of, for example, infant formulas, infant cereals, clinical infant nutritional products, toddler formulas, or as dietary supplements or health functional food for children, adults, elderly or lactating women, both as synthetically composed and naturally occurring compounds and salts thereof. Likewise, the compounds are also of interest in the medicinal industry for the production of therapeutics. In the human milk oligosaccharides the sialic acid residue is always linked to the terminal 3-O— and/or 6-O— position(s) of D-galactose and/or to the 6-O positions of N-acetylglucosamine building blocks via α-glycosidic linkage, whereas the fucose moiety is attached to the galactose of the lacto-N-biosyl group with 1-2 interglycosidic linkage and/or to the N-acetyl-glucosamine of the lacto-N-biosyl group with 1-4 interglycosidic linkage and/or to the N-acetyl-glucosamine of the N-acetyl-lactosaminyl group with 1-3 interglycosidic linkage, and/or to the galactose of the lactosyl group with 1-2 interglycosidic linkage and/or to the glucose of the lactosyl group with 1-3 interglycosidic linkage, in all the cases via α-glycosidic bond.

Desialo- and defuco-human milk oligosaccharides in suitably protected form as compound C—OH intends to mean di- or oligosaccharides such as lactose, 3-fucosyllactose, 3′-sialyllactose, 2′-fucosyllactose, lacto-N-tetraose, lacto-N-neotetraose, fucosyllacto-N-tetraoses (lacto-N-fucopentaoses), monosialyllacto-N-tetraoses (LST a, LST b, LST c), lacto-N-hexaoses, lacto-N-neohexaoses, monofucosyl-lacto-N-hexaoses, monofucosyl-lacto-N-neohexaoses, monosialyl-lacto-N-hexaoses, monosialyl-lacto-N-neohexaoses, monofucosyl-monosialyllacto-N-neohexaoses, monofucosyl-monosialyllacto-N-hexaoses, etc., that is derivatives of natural sialylated and/or fucosylated human milk oligosaccharides (see above) in which at least one sialyl or fucosyl residue is not present. Desialo-human milk oligosaccharide in suitably protected form as compound C—OH intends to mean di- or oligosaccharides such as lactose, 3-fucosyllactose, lacto-N-tetraose, fucosyllacto-N-tetraose, monosialyllacto-N-tetraose, lacto-N-fucopentaose, lacto-N-hexaose, monofucosyl-lacto-N-hexaose, monofucosyl-lacto-N-neohexaose, monofucosyl-monosialyllacto-N-neohexaose, etc., that is sialylated human milk oligosaccharides (see above) in which at least one sialyl residue is not present. The functional groups in compounds C—OH are protected except for the OH-group to be coupled. In exceptional cases they may contain 1 or 2 additional free hydroxyl groups, preferably whose reactivity is much diminished than of that to be coupled, e.g. due to steric hindrance. The protective groups on compound C—OH may be identical, similar or different to those present in the donor of general formulae I, IIA, IIB, IIIA or IIIB. Such masking groups are mentioned above.

The coupled products so obtained are in fact protected/partially protected sialylated and/or fucosylated human milk oligosaccharides, such as protected/partially protected sialylated human milk oligosaccharides. They can be subjected to remove the protective groups present. Removal of the masking groups can be carried out in one step or more consecutive steps. It is within the skilled person competence to select the appropriate reagent(s) and condition(s) for this purpose. Sialylated and fucosylated human milk oligosaccharides can be isolated from the reaction mixture using conventional work-up procedures both in solid form such as amorphous/freeze dried/spray dried or crystalline form and in liquid form as syrup or concentrated aqueous solution.

In an especially preferred embodiment the sialylated or fucosylated human milk oligosaccharide is selected from 6′-sialyllactose, 3′-sialyllactose, 2′-fucosyllactose, 3-fucosyllactose, 2′,3-difucosyllactose, 3′-sialyl-3-fucosyllactose, sialyllacto-N-tetraoses (LST a, LST b, LST c), sialyl-fucosyllacto-N-tetraoses (FLST a, FLST b, FLST c), lacto-N-fucopentaoses (LNFP I, LNFP II, LNFP III, LNFP V), lacto-N-difucohexaoses (LNDFH I, LNDFH II, LNDFH III) and disialyllacto-N-tetraose, more preferably from 6′-sialyllactose, 3′-sialyllactose, 2′-fucosyllactose, 3-fucosyllactose, 2′,3-difucosyllactose and 3′-sialyl-3-fucosyllactose.

In another especially preferred embodiment the sialylated human milk oligosaccharide is selected from 6′-sialyllactose, 3′-sialyllactose, 3′-sialyl-3-fucosyllactose, sialyllacto-N-tetraoses, sialyl-fucosyllacto-N-tetraoses and disialyllacto-N-tetraose, more preferably from 6′-sialyllactose and 3′-sialyllactose.

According to another embodiment, the synthesis of a mixture of sialylated human milk oligosaccharides is performed, characterized in that the said synthesis comprises at least the step of: coupling a compound of general formula IIIA with a mixture comprising two or more desialo-human milk oligosaccharide in protected form. Similarly, the synthesis of a mixture of fucosylated human milk oligosaccharides is performed, characterized in that the said synthesis comprises at least the step of: coupling a compound of general formula IIIB with a mixture comprising two or more defuco-human milk oligosaccharide in protected form.

The mixture of coupled products so obtained is in fact a mixture of protected sialylated human milk oligosaccharides or a mixture of protected fucosylated human milk oligosaccharides. They can be subjected to remove the protective groups present. Removal of the masking groups can be carried out in one step or more consecutive steps, preferably wherein the final step is a step of catalytic hydrogenolysis, cf. above. It is within the skilled person competence to select the appropriate reagent(s) and condition(s) for this purpose. The mixture of sialylated human milk oligosaccharides or the mixture of protected fucosylated human milk oligosaccharides can be isolated from the reaction mixture using conventional work-up procedures both in solid form such as amorphous/freeze dried/spray dried or crystalline form and in liquid form as syrup or concentrated aqueous solution.

Hence, the invention also provides a process for the synthesis of a mixture of sialylated human milk oligosaccharides, wherein a compound of general formula IIIA

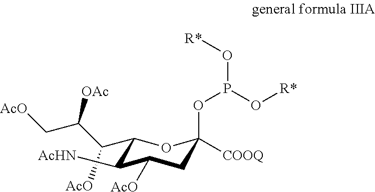

wherein Q is selected from C₁₋₆ alkyl and benzyl, preferably methyl, and R is phenyl optionally substituted with alkyl, alkoxy and/or halogen, preferably methyl, methoxy and/or bromo, is coupled with two or more protected desialo human milk oligosaccharides, followed by deprotection to give a mixture of the sialylated human milk oligosaccharides. Preferably, the mixture of sialylated human milk oligosaccharides comprises at least two sialylated human milk oligosaccharides selected from 6′-sialyllactose, 3′-sialyllactose, 3′-sialyl-3-fucosyllactose, sialyllacto-N-tetraoses, sialyl-fucosyllacto-N-tetraoses and disialyllacto-N-tetraose. In some embodiments, the coupling with two or more protected desialo human milk oligosaccharides results in a mixture of at least two compounds selected from compounds of general formulae 1 and 2 as specified hereinabove.

The invention also provides a process for the synthesis of a mixture of fucosylated human milk oligosaccharides, wherein a compound of general formula IIIB

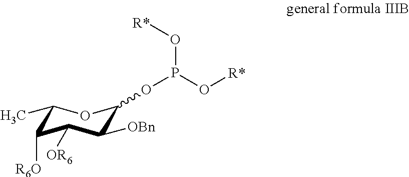

wherein R is phenyl optionally substituted with alkyl, alkoxy and/or halogen, preferably methyl, methoxy and/or bromo, and R₂ is benzyl, acetyl or benzoyl optionally substituted with chloro, is coupled with two or more protected defuco human milk oligosaccharides, followed by deprotection to give a mixture of the fucosylated human milk oligosaccharides. Preferably, the mixture of fucosylated human milk oligosaccharides comprises at least two fucosylated human milk oligosaccharides selected from 2′-fucosyllactose, 3-fucosyllactose, 2′,3-difucosyllactose, 3′-sialyl-3-fucosyllactose, sialyl-fucosyllacto-N-tetraoses (FLST a, FLST b, FLST c), lacto-N-fucopentaoses (LNFP I, LNFP II, LNFP III, LNFP V), and lacto-N-difucohexaoses (LNDFH I, LNDFH II, LNDFH III). In some embodiments, the coupling with two or more protected defuco human milk oligosaccharides results in a mixture of at least two compounds selected from compounds of general formulae 1 and 2 as specified hereinabove

In both of the above instances, the deprotection preferably involves catalytic hydrogenolysis. According to a preferred realization the mixture of sialylated human milk oligosaccharides comprises at least two sialylated human milk oligosaccharides selected from 6′-sialyllactose, 3′-sialyllactose, 3′-sialyl-3-fucosyllactose, sialyllacto-N-tetraoses (LST a, LST b, LST c), sialyl-fucosyllacto-N-tetraoses (FLST a, FLST b, FLST c) and disialyllacto-N-tetraose, whereas the mixture of protected fucosylated human milk oligosaccharides comprises at least two fucosylated human milk oligosaccharides selected from 2′-fucosyllactose, 3-fucosyllactose, 2′,3-difucosyllactose, 3′-sialyl-3-fucosyllactose, sialyl-fucosyllacto-N-tetraoses (FLST a, FLST b, FLST c), lacto-N-fucopentaoses (LNFP I, LNFP II, LNFP III, LNFP V) and lacto-N-difucohexaoses (LNDFH I, LNDFH II, LNDFH III). According to another preferred realization the mixture of sialylated human milk oligosaccharides comprises at least two sialylated human milk oligosaccharides selected from 6′-sialyllactose, 3′-sialyllactose, 3′-sialyl-3-fucosyllactose, sialyllacto-N-tetraoses, sialyl-fucosyllacto-N-tetraoses and disialyllacto-N-tetraose.

### Preparation Using Enzymatic Methods

Mixture or blend of compounds of general formulae 1 and 2 can be produced by the following steps:

a) providing at least one fucosyl, sialyl, N-acetyllactosaminyl or lacto-N-biosyl donor,
b) providing at least one acceptor selected from lactose R-glycoside, LNT R-glycoside and LNnT R-glycoside, wherein R is as defined above,
c) preparing a blend from compounds provided by steps a) and b);
d) adding at least one enzyme comprising a transglycosidase activity and/or a glycosynthase activity to the blend of step c) thereby forming a mixture;
e) incubating the mixture obtained according to step d); and
f) optionally repeating any of steps a) to d), preferably with the mixture obtained according to step e).

According to step a) at least one fucosyl, sialyl, N-acetyllactosaminyl or lacto-N-biosyl donor is provided and according to step b) at least one acceptor is provided. In the context of the present invention, the term “fucosyl, sialyl, N-acetyllactosaminyl or lacto-N-biosyl donor” is preferably understood as compounds, which provide or transfer a fucosyl, sialyl, N-acetyllactosaminyl or lacto-N-biosyl moiety in a chemical reaction, e.g. an enzymatic glycosylation, to a further compound, preferably an acceptor. Such “fucosyl, sialyl, N-acetyllactosaminyl or lacto-N-biosyl donors” are advanced or activated glycosyl compounds like e.g. glycosyl fluorides, glycosyl azides, optionally substituted phenyl glycosides, optionally substituted pyridinyl glycosides, optionally substituted 3-oxo-(2H)-furan-4-yl glycosides, optionally substituted 1,3,5-triazinyl glycosides or 4-methylumbelliferyl glycosides.

According to step c) a blend prepared from compounds provided by steps a) and b). Preferably, such a blend according to step c) represents a blend of one, two, three, four, five, one to five, three to ten, five to ten or even more preferably different compounds as defined according to step a) and one, two, three, four, five, one to five, three to ten, five to ten or even more preferably different compounds as defined according to step b).

In step d) of the inventive method for generating human milk oligosaccharides (HMOs) at least one enzyme comprising a transglycosidase activity and/or a glycosynthase activity is added to the blend obtained according to step c) of the inventive method, thereby forming a mixture.

In step d) at least one enzyme comprising transglycosidase activity and/or glycosynthase activity is added, preferably at least two (preferably different), three (preferably different), four (preferably different), five (preferably different), two to five (preferably different), two to ten (preferably different), two to twenty (preferably different), five to ten (preferably different) or even more preferably different enzymes comprising transglycosidase activity and/or glycosynthase activity.

Enzymes suitable in step d) typically comprise at least one enzyme comprising a transglycosidase activity and/or a glycosynthase activity, preferably selected from enzymes having, e.g. transglycosidase activity and/or a glycosynthase activity, e.g. having a fucosidase or trans-fucosidase, a sialidase (neuraminidase) or trans-sialidase (transneuraminidase), a lacto-N-biosidase or trans-lacto-N-biosidase and/or a N-acetyllactosaminidase or trans-N-acetyllactosaminidase activity, or any further enzyme having such an activity. Even more preferably, enzymes suitable in step d) may be selected from wild type or mutated glycosidases or transglycosidases, preferably wild type or mutated glycosidases or transglycosidases having a fucosidase or trans-fucosidase, a sialidase (neuraminidase) or trans-sialidase (transneuraminidase), a lacto-N-biosidase or trans-lacto-N-biosidase and/or a N-acetyllactosaminidase or trans-N-acetyllactosaminidase activity, or preferably having α-trans-fucosidase, α-trans-sialidase, β-trans-Lacto-N-biosidase and/or β-trans-N-acetyllactosaminidase activity.

Enzymes suitable in step d) furthermore may be obtained from any genus known to a skilled person, to express or secrete at least one enzyme as defined above, e.g. an enzyme having a transglycosidase activity and/or a glycosynthase activity, preferably an enzyme having a fucosidase or trans-fucosidase, a sialidase (neuraminidase) or trans-sialidase (transneuraminidase), a lacto-N-biosidase or trans-lacto-N-biosidase and/or a N-acetyllactosaminidase or trans-N-acetyllactosaminidase activity, or preferably having α-trans-fucosidase, α-trans-sialidase, β-trans-Lacto-N-biosidase and/or β-trans-N-acetyllactosaminidase activity, or any further enzyme having such an activity. Even more preferably, such enzymes suitable in step d) may be obtained from bacteria selected from Bacillus, Bifidobacterium, Lactobacillus, Leuconostoc, Lactococcus, Streptococcus, Streptomyces, Sulfolobus, Thermotoga, or Trypanosoma.

The mixture is incubated in a further step e). Such an incubation advantageously allows generating a multiplicity of different compound characterized by general formulae 1 and 2 defined above. Generation of such a multiplicity of different compounds is based on the use of enzymes with different activities during step d) but also on the use of diverse donors and acceptors according to steps a) and b), preferably as a blend as already outlined in step c). Utilizing this approach, the method advantageously allows varying the possible number and type of oligosaccharides obtainable by the synthesis in a simple and cost efficient manner. The use of enzymes furthermore allows carrying out the preparation of various derivatives in a stereoselective manner. Generation of compounds preferably occurs by transferring N-acetyllactosaminyl moieties, lacto-N-biosyl moieties, fucosyl moieties, silyl moieties, by forming new bonds at desired positions of the molecule, etc., in a well defined manner to obtain a mixture of various human milk oligosaccharides R-glycosides.

Incubation according to step e) occurs with a concentration of (each of the) enzymes in a concentration of 1 mU/L to 1,000 U/L, preferably 10 mU/L to 100 U/L, when the activity capable of forming 1 μmol of specific product for a defined protein starting from a defined educt is defined as 1 unit (U), e.g. for a glycotransferase the production of a glycose-containing complex carbohydrate at 37° C. in 1 minute. The activity of each enzyme as defined herein may be assessed with respect to its naturally occurring or engineered substrate.

The incubation according to step e) may be carried out in a reaction medium, preferably an aqueous medium, comprising the mixture obtained according to step d) and optionally water; a buffer such as a phosphate buffer, a carbonate buffer, an acetate buffer, a borate buffer, a citrate buffer and a tris buffer, or combinations thereof; alcohol, such as methanol and ethanol; ester such as ethyl acetate; ketone such as acetone; amide such as acetamide; and the like.

Furthermore, the incubation according to step e) may be carried out in a reaction medium as defined above, wherein optionally a surfactant or an organic solvent may be added, if necessary. Any surfactant capable of accelerating the formation of a complex carbohydrate as defined according to the present invention as a possible product of the invention can be used as the surfactant. Examples include non-ionic surfactants such as polyoxyethylene octadecylamine (e.g., Nymeen S-215, manufactured by Nippon Oil & Fats); cationic surfactants, such as cetyltrimethylammonium bromide and alkyldimethyl benzylammoniumchloride (e.g., Cation F2-40E, manufactured by Nippon Oil & Fats); anionic surfactants such as lauroyl sarcosinate; tertiary amines such as alkyldimethylamine (e.g., Tertiary Amine FB, manufactured by Nippon Oil & Fats); and the like, which are used alone or as a mixture of two or more. The surfactant may be used generally in a concentration of 0.1 to 50 g/l. The organic solvent may include xylene, toluene, fatty acid alcohol, acetone, ethyl acetate, and the like, which may be used in a concentration of generally 0.1 to 50 ml/l.

The incubation according to step e) may be furthermore carried out in a reaction medium as defined above, preferably having a pH 3 to 10, pH 5 to 10, preferably pH 6 to 8.

The incubation according to step e) may be furthermore carried out at a temperature of about 0° C. to about 100° C., preferably at a temperature of about 10 to about 50° C., e.g. at a temperature of about 20° C. to about 50° C. In the reaction medium, inorganic salts, such as MnCl₂ and MgCl₂, may be added, if necessary.

The incubation according to step e) may be carried out in a bioreactor. The bioreactor is preferably suitable for either a continuous mode or a discontinuous mode.

Alternatively, a mixture or blend of compounds of general formulae 1 and 2 can be produced by the following steps:
a) providing at least one compound or a mixture of compounds selected from:
   optionally sialylated and/or fucosylated lactose derivative of general formula 2:

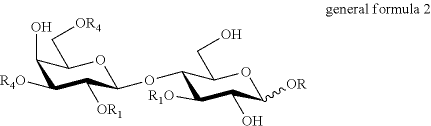

wherein
R is a group removable by hydrogenolysis,
R₁ independently of each other is fucosyl or H
R₄ independently of each other is sialyl or H,
or salts thereof,
preferably provided the compound of general formula 2 is not R-glycoside of lactose, if provided alone;
a lacto-N-tetraose (LNT) derivative of following formula:

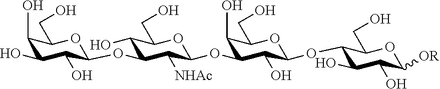

wherein R is a group removable by hydrogenolysis; and
a lacto-N-neotetraose (LNnT) derivative of following formula:

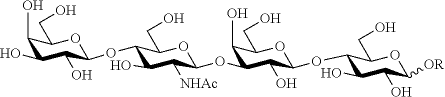

wherein R is a group removable by hydrogenolysis;
b) adding at least one enzyme comprising a transglycosidase activity to the at least one compound or a mixture of compounds provided according to step a);
c) incubating the mixture obtained according to step b);
d) optionally repeating any of steps a) to c), preferably with the mixture obtained according to step c).

According to step a), at least one compound or a mixture of compounds is provided. Such a mixture of compounds is preferably to be understood as a mixture of at least two, three, four, five, one to five, five to ten, one to ten, two to ten, two to twenty, three to twenty, four or even five to twenty, or even more of the same or different compounds as generally defined according to any of the compounds of step a). Accordingly, such at least one compound or a mixture at least two, three, four, five, one to five, five to ten, one to ten, two to ten, two to twenty, three to twenty, four or even five to twenty, or even more of the same or different compounds as generally defined according to any of the compounds of step a) may be selected without restriction from any of the compounds as defined according to formula 2 or LNT derivatives or LNnT derivatives as defined above.

Components as defined according to step a), particularly components as defined according to formula 2 or LNT derivatives or LNnT derivatives as defined above, may serve as a donor or as an acceptor in the inventive method for diversification of human milk oligosaccharides (HMOs) or precursors thereof. In the context of the present invention, the term “donor” is preferably understood as a compound, which provides a specific moiety in a chemical reaction, e.g. a nucleophilic or electrophilic substitution reaction, to a further compound, preferably an acceptor. Likewise, the term “acceptor” is preferably understood as a compound, which receives a specific moiety in a chemical reaction, e.g. nucleophilic or electrophilic substitution reaction, to a further compound, preferably a donor.

In step b) at least one enzyme comprising transglycosidase activity is added, preferably at least two (preferably different), three (preferably different), four (preferably different), five (preferably different), two to five (preferably different), two to ten (preferably different), two to twenty (preferably different), five to ten (preferably different) or even more preferably different enzymes comprising transglycosidase activity.

Enzymes suitable in step b) typically comprise at least one enzyme comprising a transglycosidase activity, preferably selected from enzymes having, e.g. a fucosidase or trans-fucosidase, a sialidase (neuraminidase) or trans-sialidase (transneuraminidase), a lacto-N-biosidase or trans-lacto-N-biosidase and/or a N-acetyllactoaminidase or trans-N-acetyllactoaminidase activity, or any further enzyme having such an activity. Even more preferably, enzymes suitable in step b) of the inventive method for diversification of human milk oligosaccharides (HMOs) may be selected from the group consisting of wild type or mutated glycosidases or transglycosidases, preferably wild type or mutated glycosidases or transglycosidases having a fucosidase or trans-fucosidase, a sialidase (neuraminidase) or trans-sialidase (transneuraminidase), a lacto-N-biosidase or trans-lacto-N-biosidase and/or a N-acetyllactoaminidase or trans-N-acetyllactoaminidase activity, or preferably having α-trans-fucosidase, α-trans-sialidase, β-trans-lacto-N-biosidase and/or β-trans-N-acetyllactosaminidase activity.

Enzymes suitable in step b) further may be obtained from any genus known to a skilled person, to express or secrete at least one enzyme as defined above, e.g. an enzyme having a transglycosidase activity, preferably an enzyme having a fucosidase or trans-fucosidase, a sialidase (neuraminidase) or trans-sialidase (transneuraminidase), a lacto-N-biosidase or trans-lacto-N-biosidase and/or a N-acetyllactoaminidase or trans-N-acetyllactoaminidase activity, or preferably having α-trans-fucosidase, α-trans-sialidase, β-trans-lacto-N-biosidase and/or β-trans-N-acetyllactosaminidase activity, or any further enzyme having such an activity. Even more preferably, such enzymes suitable in step b) of the inventive method for diversification of human milk oligosaccharides (HMOs) may be obtained from bacteria selected from Bacillus, Bifidobacterium, Lactobacillus, Leuconostoc, Lactococcus, Streptococcus, Streptomyces, Sulfolobus, Thermotoga, or Trypanosoma.

In a further step c), the mixture containing at least one compound as defined according to step a) or a mixture thereof and at least one enzyme as added according to step b) are preferably incubated to allow diversification of human milk oligosaccharide (HMO) derivatives via enzymatic means using the at least one enzyme comprising a transglycosidase activity as defined herein. Incubation step according to step c) can be performed as described above.

The addition of enzymes in step b) and without prejudice any of steps a), b) and c) may be repeated according to an optional step d). Particularly preferably, any of steps a) to c) may be repeated, preferably with the mixture obtained according to step c). Preferably, this mixture has already been incubated and thus processed with at least one compound as defined herein for step a) and at least one enzyme as defined herein according to step b). Such a staggered proceeding may allow within multiple rounds the rational diversification of a defined set of educts to a limited set of compounds in a controllable manner. Adding specific compounds as defined according to step a) and different proteins as defined according to step b) in a predetermined order may also provide for a rational exclusion of specific components. To obtain such a variety, the compounds and/or enzymes may be added preferably simultaneously or sequentially, more preferably compounds and/or enzymes may be added simultaneously in one step and/or sequentially in different steps.

Alternatively, a compound or a mixture of compounds as defined herein for step a) and at least one enzyme as defined herein according to step b) may be incubated in one step, preferably when all compounds provided simultaneously. Such a proceeding may also be preferable, as it may lead to the largest variety of diversified compounds of general formulae 1 and 2.

Mixture Obtained after Hydrogenolysis

The method of catalytic hydrogenolysis of compounds of general formulae 1 and 2 provides a mixture of human milk oligosaccharides (HMOs), the single compounds of which may be defined according to general formulae 3 and 4 respectively.

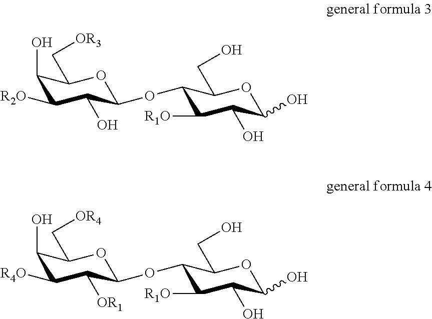

R₁ is independently fucosyl or H,
R₂ is selected from N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₃ is H or N-acetyl-lactosaminyl group optionally substituted with a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₄ is independently sialyl or H,
and salts thereof,
with the proviso that at least one of R₁ or R₄ is not H in general formula 4.

HMO components produced by hydrogenolysis as defined above, particularly components as defined under general formulae 3a, 3b and 4

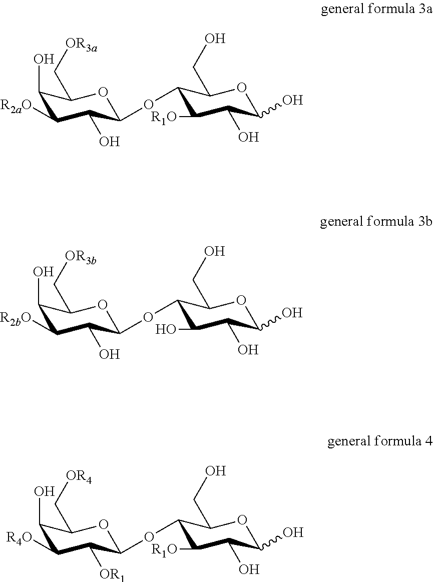

wherein R₁ and R₄ are as defined above,
R₂, is N-acetyl-lactosaminyl group optionally substituted with a glycosyl residue comprising one N-acetyl-lactosaminyl and/or one lacto-N-biosyl group; any N-acetyl-lactosaminyl and lacto-N-biosyl group can be substituted with one or more sialyl and/or fucosyl residue,
R₃ₐ is H or N-acetyl-lactosaminyl group optionally substituted with a lacto-N-biosyl group; any N-acetyl-lactosaminyl and lacto-N-biosyl group can be substituted with one or more sialyl and/or fucosyl residue,
R_{2b} is lacto-N-biosyl group optionally substituted with sialyl and/or fucosyl residue,
R_{3b} is H or N-acetyl-lactosaminyl group optionally substituted with one or two N-acetyl-lactosaminyl and/or one lacto-N-biosyl group; any N-acetyl-lactosaminyl and lacto-N-biosyl group can be substituted with one or more sialyl and/or fucosyl residue.

More preferably, compounds are obtained after hydrogenolysis, wherein
the N-acetyl-lactosaminyl group in the glycosyl residue of R₂ₐ in general formula 3a is attached to the another N-acetyl-lactosaminyl group with 1-3 interglycosidic linkage,
the lacto-N-biosyl group in the glycosyl residue of R₂ₐ in general formula 3a is attached to the N-acetyl-lactosaminyl group with 1-3 interglycosidic linkage,
the lacto-N-biosyl group in the glycosyl residue of R₃ₐ in general formula 3a is attached to the N-acetyl-lactosaminyl group with 1-3 interglycosidic linkage,
the N-acetyl-lactosaminyl group in the glycosyl residue of R_{3b} in general formula 3b is attached to the another N-acetyl-lactosaminyl group with 1-3 or 1-6 interglycosidic linkage,
the lacto-N-biosyl group in the glycosyl residue of R_{3b} in general formula 3b is attached to the N-acetyl-lactosaminyl group with 1-3 interglycosidic linkage.

Even more preferable compounds obtained after hydrogenolysis are compounds of general formula 3a and 3b, wherein general formula 3a represents lacto-N-neotetraose, para-lacto-N-hexaose, para-lacto-N-neohexaose, lacto-N-neohexaose, para-lacto-N-octaose and lacto-N-neooctaose optionally substituted with one or more sialyl and/or fucosyl residue, and general formula 3b represents lacto-N-tetraose, lacto-N-hexaose, lacto-N-octaose, iso-lacto-N-octaose, lacto-N-decaose and lacto-N-neodecaose optionally substituted with one or more sialyl and/or fucosyl residue.

Particularly preferable compounds as products of hydrogenolysis are wherein
the fucosyl residue attached to the N-acetyl-lactosaminyl and/or the lacto-N-biosyl group is linked to
the galactose of the lacto-N-biosyl group with 1-2 interglycosidic linkage and/or
the N-acetyl-glucosamine of the lacto-N-biosyl group with 1-4 interglycosidic linkage and/or
the N-acetyl-glucosamine of the N-acetyl-lactosaminyl group with 1-3 interglycosidic linkage,
the sialyl residue attached to the N-acetyl-lactosaminyl and/or the lacto-N-biosyl group is linked to
the galactose of the lacto-N-biosyl group with 2-3 interglycosidic linkage and/or
the N-acetyl-glucosamine of the lacto-N-biosyl group with 2-6 interglycosidic linkage and/or
the galactose of the N-acetyl-lactosaminyl group with 2-6 interglycosidic linkage.

The most preferable compounds obtained in hydrogenolysis represent naturally occurring HMOs having a lactose, LNT or LNnT core, and are selected from the group of: 2′-fucosyllactose, 3-fucosyllactose, 2′,3-difucosyllactose, 3′-sialyllactose, 6′-sialyllactose, 3′-sialyl-3-fucosyllactose, lacto-N-tetraose, lacto-N-neotetraose, LNFP-I, LNFP-II, LNFP-III, LNFP-V, LST-a, LST-b, LST-c, FLST-a, FLST-b, FLST-c, LNDFH-I, LNDFH-II, LNDFH-III, DS-LNT, FDS-LNT I and FDS-LNT II.

The advantage of the present method resides on providing HMO blend whose composition may be very close to that of the natural mother's milk. As the catalytic hydrogenolysis proceeds practically without by-product formation and almost quantitatively, the composition of the starting mixture comprising compounds of general formulae 1 and 2 is directly proportional to the composition of the produced HMOs according to general formulae 3 and 4. Thus careful tuning of the starting materials may lead compositions analogue to human milk.

Thus, in a preferred embodiment of the method a mixture of individual compounds characterized by general formulae 1 and 2 defined above comprising 0-100% of compounds containing one or more sialyl residue but devoid of fucosyl residue, 0-100% of compounds containing one or more fucosyl residue but devoid of sialyl residue, 0-100% of compounds containing one or more sialyl and one or more fucosyl residue and 0-100% of compounds devoid of sialyl and fucosyl residue, is subjected to catalytic hydrogenolysis to obtain a mixture of individual compounds characterized by general formulae 3 and 4 defined above comprising 0-100% of compounds containing one or more sialyl residue but devoid of fucosyl residue, 0-100% of compounds containing one or more fucosyl residue but devoid of sialyl residue, 0-100% of compounds containing one or more sialyl and one or more fucosyl residue and 0-100% of compounds devoid of sialyl and fucosyl residue.

The mixture of human milk oligosaccharides made by the present invention can be used in a consumable product, in particular pharmaceutical and nutritional use. The mixture of human milk oligosaccharides is particularly effective in the education and/or maturation of the immune system of neonatal infants, and has preventive effect against secondary infections following viral infections such as influenza.

The mixture of human milk oligosaccharides as a prebiotic enhances the beneficial effects and efficiency of probiotics, such as, but not limited to Lactobacillus and Bifidobacterium species, in promoting the development of an early bifidogenic intestinal microbiota in infants, in reducing the risk of development or allergy and/or asthma in infants, in preventing and treating pathogenic infections in such as diarrhoea in infants. Furthermore additional probiotics can be added, e.g. lacto bacteria, Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g. palm olein, sunflower oil, safflower oil) and vitamins and minerals essential in a daily diet can also be further added.

The mixture of human milk oligosaccharides can be added to a pharmaceutically acceptable carriers such as, but not limited to additives, adjuvants, excipients and diluents (water, gelatine, talc, sugars, starch, gum arabic, vegetable gums, vegetable oils, polyalkylene glycols, flavouring agents, preservatives, stabilizers, emulsifying agents, lubricants, colorants, fillers, wetting agents, etc.). Suitable carriers are described in the most recent edition of Remington's Pharmaceutical Sciences, a standard reference text in the field. When the mixture of human milk oligosaccharides is added to the pharmaceutically acceptable carriers a dosage form in the form of for example, but not limited to tablets, powders, granules, suspensions, emulsions, infusions, capsules, injections, liquids, elixirs, extracts and tincture can be formed.

In a further embodiment the consumable product can be nutritional formulation such as foods, drinks or feeds, containing edible micronutrients, vitamins and minerals. The amounts of such ingredient may vary depending on whether the consumable product is intended for use with healthy infants, children, adults or subjects having specialized needs (e.g. suffering from metabolic disorders). Micronutrients include for example edible oils, fats or fatty acids (such as coconut oil, soy-bean oil, monoglycerides, diglycerides, palm olein, sunflower oil, fish oil, linoleic acid, linolenic acid etc.), carbohydrates (such as glucose, fructose, sucrose, maltodextrin, starch, hydrolyzed cornstarch, etc.) and proteins from casein, soy-bean, whey or skim milk, or hydrolysates of these proteins, but protein from other source (either intact or hydrolysed) may be used as well. Vitamins may be chosen such as vitamin A, 61, 62, 65, 66, B12, C, D, E, H, K, folic acid, inositol and nicotinic acid. The nutritional formula may contain the following minerals and trace elements: Ca, P, K, Na, Cl, Mg, Mn, Fe, Cu, Zn, Se, Cr or I.

In a further embodiment the consumable product can be infant formula. In the context of the present invention, the term “infant formula” preferably means a foodstuff intended for particular nutritional use by infants during the first 4-6 months or even 4 to 12 months of life and satisfying by itself the nutritional requirements of infants. It may contain one or more probiotic Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g. palm olein, sunflower oil, safflower oil) and vitamins and minerals essential in a daily diet.

In a further embodiment the consumable product can be a food supplement. Such a food supplement preferably contains ingredients as defined for nutritional food above, e.g. vitamins, minerals, trace elements and other micronutrients, etc. The food supplement may be for example in the form of tablets, capsules, pastilles or a liquid. The supplement may contain conventional additives selected from but not limited to binders, coatings, emulsifiers, solubilising agents, encapsulating agents, film forming agents, adsorbents, carriers, fillers, dispersing agents, wetting agents, jellifying agents, gel forming agents, etc.

In a further embodiment the consumable product can be digestive health functional food as the administration of compounds as prepared according to the present invention, more preferably a diversified blend of HMOs, as may be prepared by the inventive method, provides a beneficial effect on digestive health. Digestive health functional food is preferably a processed food used with intention to enhance and preserve digestive health by utilizing compounds as prepared according to the present invention, more preferably a diversified blend of HMOs, as may be prepared by the inventive method, as physiologically functional ingredients or components in forms of tablet, capsule, powder, etc. Different terms such as dietary supplement, nutraceutical, designed food, health product may also be used to refer to digestive health functional food.

### EXAMPLES

### Example 1

Mixtures of compounds by general formula 1 and 2 were dissolved in a protic solvent such as water or alcohol or mixtures thereof (4 volumes) and 10% Pd on charcoal ( 1/10 weight compared to the weight of starting carbohydrates) was added, and the pH of the solution was adjusted to 4-6 by addition of an acid such as, but not limited to HCl, sialic acid or citric acid to form a suspension. The suspension was treated with hydrogen gas (5-6 bar) at 54° C. whilst being stirred. The hydrogenolysis was complete within 2-3 hours. The resulting suspension was filtered to provide a solid product. The final product is washed with warm water and died to provide the mixture of the HMOs

### Example 2

A mixture of 1-O-benzyl-LNT (0.3 g), 1-O-benzyl-LNnT (0.3 g)), 1-O-benzyl-2′-FL (0.3 g) and 1-O-benzyl-6′-SL (0.3 g) was dissolved in 4.8 ml of water to form a solution. To the solution 0.12 g of 10% Pd/C was added, followed by the addition of concentrated HCl until the pH of the solution is pH 4. The suspension was treated with hydrogen gas (5-6 bar) at 54° C. whilst being stirred. The resulting suspension was filtered to provide a solid product of a mixture of HMOs of LNT/LNnT/2′-FL/6′-SL.

### Example 3

### Preparation of Donors

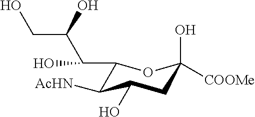

Method A: A mixture of anhydrous sialic acid (100 g, 323 mmol) and dried Amberlite IR-120 (H⁺) ion exchange resin (100 g) in MeOH (1500 mL) was stirred for 15 hours at RT. The ion exchange resin was filtered off and washed with MeOH (2×100 mL). The washes were combined with the filtrate and concentrated to 300 mL. The concentrated residue crystallized upon seeding at RT. The crystals were collected by filtration giving 73.8 g (71%) sialic acid methyl ester. The mother liquor was concentrated (10.5 g) and recrystallized from MeOH (30 mL) to yield 7.4 g (7%) sialic acid methyl ester. Total yield 81.2 g (78%).

Method B: To a suspension of anhydrous sialic acid (100 g, 323 mmol) in MeOH (1200 ml) 8% HCl in MeOH (50 ml) was added and the reaction mixture was stirred for 6 hours at RT. The reaction mixture was neutralized with triethylamine (15 ml) and the clear solution was concentrated to 270 mL. The concentrated residue crystallized upon seeding at RT for 2 hours. The solid was collected by filtration yielding 104.9 g (100%). ¹H NMR (D₂O) δ in ppm: 1.89 (dd, 1H, J=13.0 Hz, J=11.4 Hz); 2.03 (s, 3H); 2.28 (dd, 1H, J=13.0 Hz, J=4.7 Hz); 3.52 (dd, 1H, J=8.9 Hz, J=3.0 Hz); 3.59 (dd, 1H, J=11.6 Hz, J=6.1 Hz); 3.71 (ddd, J=8.9 Hz, J=2.5 Hz, J=11.6 Hz); 3.82 (dd, 1H, J=11.6 Hz, J=2.5 Hz); 3.82 (s, 3H); 3.90 (dd, 1H, J=10.1 Hz, J=10.0 Hz); 3.98-4.10 (m, 2H, H-6, H-4).

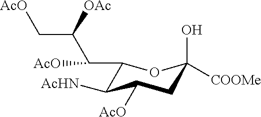

Method A: A suspension of sialic acid methyl ester (50 g, 155 mmol) and acetic anhydride (73 ml, 775 mmol) in DCM (175 mL) was stirred at RT and 70% perchloric acid (1 mL) was then added dropwise within 30 minutes. During the addition the temperature of the mixture increased until reflux. The reaction mixture was stirred at reflux for 2.5 h, and after this time MeOH (7.5 ml, 185 mmol) was added dropwise and the reaction mixture was stirred for a further hour at RT. The reaction mixture was diluted with DCM (175 mL) and washed with water (3×50 mL). The combined water phases were extracted with DCM (2×100 mL). The combined organic phases were washed with saturated NaHCO₃ (2×100 mL) and evaporated. The residue (59.6 g) was dissolved in iBuOAc at 50° C. and the mixture was cooled down to RT and let overnight complete the crystallization. The solid was collected by filtration yielding 39.2 g (52%) of tetraacetyl sialic acid methyl ester.

Method B: To a mixture of sialic acid methyl ester (60.8 g, 188 mmol) and acetic anhydride (89 ml, 940 mmol) in DCM (220 mL), perchloric acid 70% (1.22 mL) was added dropwise within 30 minutes. During the addition the temperature of the mixture increased until reflux (47° C.). The reaction mixture is stirred at reflux for 2.5 h. Subsequently, MeOH (9.2 mL, 225 mmol) was added dropwise and the reaction mixture was stirred for one additional hour at RT. The clear solution was added dropwise to a suspension of Na₂CO₃ (60.8 g; 573 mmol) in DCM and the mixture was stirred at room temperature for 2 hours. The remaining solid was removed by filtration and was washed with DCM (2×50 mL). The combined DCM phase was concentrated to 150 ml, ⁱBuOAc (150 mL) was added and the remaining DCM was removed and let crystallize overnight. The crystals were collected by filtration yielding 65 g (71%) of tetraacetyl sialic acid methyl ester. ¹H NMR (C₆D₆) δ in ppm: 1.60, 1.63, 1.70, 1.85, 1.92 (5s, 15H); 2.19 (dd, 1H, J=12.8 Hz, J=5.7 Hz); 2.25 (ddd, 1H, J=12.8 Hz, J=10.8 Hz); 3.28 (s, 3H); 4.23 (dd, 1H, J=12.4 Hz, J=7.6 Hz); 4.26 (dd, 1H); 4.54 (ddd, 1H, J=10.8 Hz); 4.78 (d, 1H, J=10.2 Hz); 5.02 (dd, 1H, J=12.4 Hz, J=2.0 Hz); 5.26 (ddd, 1H, J=10.8 Hz, J=5.7 Hz, J=10.5 Hz); 5.61 (ddd, 1H, J=2.0 Hz, J=7.6 Hz); 5.64 (dd, 1H, J=2.3 Hz, J=4.2 Hz). ¹H NMR (CDCl₃) δ in ppm: 1.91, 2.02, 2.03, 2.11, 2.15 (5s, 15H); 2.19 (dd, 1H, J=12.8 Hz, J=11.4 Hz); 3.26 (ddd, 1H, J=12.8 Hz, J=5.4 Hz); 3.86 (s, 3H); 4.03 (dd, 1H, J=12.4 Hz, J=7.5 Hz); 4.13-4.21 (m, 2H); 4.51 (dd, 1H, J=12.4 Hz, J=2.4 Hz); 5.22 (ddd, 1H, J=11.4 Hz, J=5.4 Hz, J=9.5 Hz); 5.25 (ddd, 1H, J=2.4 Hz, J=7.5 Hz, J=5.6 Hz); 5.36 (dd, 1H, J=1.5 Hz, J=5.6 Hz); 5.71 (m, 1H). ¹³C NMR: 20.65, 20.75, 20.93, 22.93, 36.11, 49.04, 53.18, 62.50, 68.3, 69.12, 71.32, 72.07, 94.84, 168.93, 170.12, 170.30, 170.72, 171.04, 171.43.

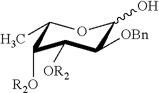

Partially protected fucose derivatives (R₂ means benzoyl or 4-chlorobenzoyl) with free anomeric OH were prepared analogously to Eisele et al. Carbohydr. Res. 306, 81 (1998).

### Example 4

### General Procedure of Preparing Glycosyl Phosphites

DCM, Et₃N (5.5 eq.) and imidazole (4.5 eq.) were placed into the flask and the suspension was cooled to 5° C. PCl₃ (1.5 eq.) was then added and the reaction mixture was stirred for 1 h at 5° C. One equivalent of 1-OH-sugar derivative (N-acetyl neuraminic acid methyl ester tetra-O-acetate, or 2-O-benzyl-3,4-di-O-benzoyl-L-fucose, or 2-O-benzyl-3,4-di-O-(4-chlorobenzoyl)-L-fucose) in DCM was added and the temperature allowed to warm up to 20° C. The mixture was stirred for 3 h at r.t. followed by the addition of the phenol derivative (3.5 eq.). The mixture was stirred for 1 h. The base was neutralized by addition of 2 N HCl solution and the mixture was stirred vigorously for 15 min. The phases were separated. The organic phase was washed with NaHCO₃ solution and with water. The organic phase was concentrated and in case of sialic acid derivatives the product was crystallized, or in case of the fucose derivatives the product was isolated after chromatography (yield: 70-80%).

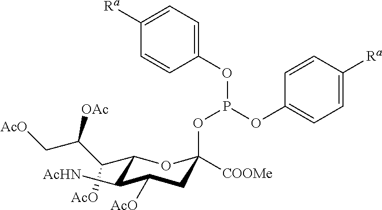

R^{a}=methyl: mp: 128.3-130.8° C. ¹H NMR (CDCl₃) δ (ppm): 1.84 (s, 3H); 1.90 (m, 3H); 2.00 (s, 3H); 2.04 (s, 3H); 2.10 (m, 4H); 2.31 (s, 6H); 2.47 (dd, 1H, J=4.9 Hz, J=13.1 Hz); 3.75 (s, 3H); 3.9 (dd, 1H, J=2.3 Hz, J=10.7 Hz); 4.1 (m, 2H); 4.55 (dd, 1H, J=12.3 Hz, J=2.4 Hz); 4.87 (d, 1H, J=10.3 Hz); 5.1 (m, 2H); 5.27 (m, 1H); 6.97-7.2 (m, 8H). ¹³C NMR (CDCl₃) δ (ppm): 20.93, 20.96, 20.99, 21.09, 21.30, 23.37, 38.16, 48.93, 53.62, 62.75, 68.25, 68.64, 72.25, 72.77, 98.84, 98.87, 121.05, 121.14, 130.41, 130.50, 133.98, 134.08, 149.33, 149.35, 167.13, 170.25, 170.29, 170.60, 170.81, 170.97.

R^{a}=methoxy: mp: 124.8-126.7° C. ¹H NMR (CDCl₃) δ (ppm): 1.84 (s, 3H); 1.88 (m, 3H); 2.00 (s, 3H); 2.01-2.07 (m, 4H); 2.09 (s, 3H); 2.44 (dd, 1H, J=4.9 Hz, J=13 Hz); 3.64 (dd, 1H, J=2.5 Hz, J=10.7 Hz); 3.77 (m, 9H); 4.07 (m, 2H); 4.55 (dd, 1H, J=12.3 Hz, J=2.3 Hz); 4.8 (d, 1H, J=10.4 Hz); 5.01 (dd, 1H, J=10.9 Hz, J=4.9 Hz); 5.08 (m, 1H); 5.22 (t, 1H, J=2.8 Hz); 6.9 (m, 4H); 7.1 (m, 4H). ¹³C NMR (CDCl₃) δ (ppm): 20.93, 20.98, 21.09, 21.30, 23.15, 38.13, 48.72, 53.71, 55.84, 62.79, 68.33, 68.52, 72.50, 72.61, 98.93, 114.95, 115.07, 122.37, 122.46, 122.62, 122.62, 144.98, 144.05, 144.08, 154.41, 167.21, 170.30, 170.33, 170.67, 170.80, 170.93.

R^{a}═H: mp: 122.3-123.9° C. ¹H NMR (CDCl₃) δ (ppm): 1.84 (s, 3H); 1.89 (m, 3H); 2.00 (s, 3H); 2.04 (s, 3H); 2.10 (m, 4H); 2.47 (dd, 1H, J=4.9 Hz, J=13.1 Hz); 3.75 (s, 3H); 3.87 (dd, 1H, J=2.4 Hz, J=10.7 Hz); 4.1 (m, 2H); 4.55 (dd, 1H, J=12.3 Hz, J=2.4 Hz); 4.80 (d, 1H, J=10.2 Hz); 5.05 (dd, 1H, J=4.9 Hz, J=10.9 Hz); 5.12 (m, 1H); 5.26 (dd, 1H, J=2.5 Hz, J=3.5 Hz); 7.1-7.25 (m, 6H); 7.31-7.41 (m, 4H). ¹³C NMR (CDCl₃) δ (ppm): 20.92, 20.98, 21.09, 21.30, 23.38, 38.12, 48.76, 53.71, 62.75, 68.29, 68.57, 72.31, 72.86, 99.03, 99.06, 121.33, 121.42, 121.45, 121.54, 124.45, 124.50, 130.05, 130.14, 151.70, 167.12, 170.25, 170.27, 170.65, 170.80, 170.97.

R^{a}=Br: ¹H NMR (CDCl₃) δ (ppm): 1.88 (s, 6H); 2.01 (s, 3H); 2.03 (s, 3H); 2.09 (m, 4H); 2.44 (dd, 1H, J=4.9 Hz, J=13.2 Hz); 3.78 (s, 3H); 3.85 (dd, 1H, J=2.6 Hz, J=10.6 Hz); 3.96-4.21 (m, 2H); 4.51 (dd, 1H, J=12.3 Hz, J=2.3 Hz); 4.94 (d, 1H, J=10.2 Hz); 5.05 (m, 2H); 5.26 (m, 1H); 7.04 (m, 4H); 7.47 (m, 4H). ¹³C NMR (CDCl₃) δ (ppm): 20.90, 20.96, 21.05, 21.28, 23.43, 38.15, 48.88, 53.92, 62.65, 68.32, 68.39, 72.32, 73.17, 99.40, 117.18, 123.01, 123.10, 123.25, 123.33, 133.02, 133.09, 150.59, 150.62, 167.09, 170.23, 170.51, 170.72, 170.97.

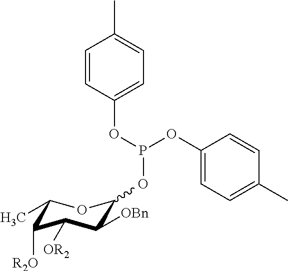

R₂=4-chlorobenzoyl, α-anomer: ¹H NMR (CDCl₃) δ (ppm): 1.12 (s, 3H, J=6.0 Hz), 2.33 (s, 6H), 4.16 (dd, 1H, J=3.0 12.0 Hz), 4.45-4.52 (m, 1H), 4.63 (d, 1H, J=12.0 Hz), 4.69 (d, 1H, J=12.0 Hz), 5.65-5.67 (m, 1H), 5.79 (dd, 1H, J=3.0 12.0 Hz), 6.15 (dd, 1H, J=3.0 9.0 Hz), 7.13-7.92 (m, 21H). ¹³C NMR (CDCl₃) δ (ppm): 15.8, 20.6 (2C), 66.2, 70.5, 72.2, 72.4, 72.7 (d, J=4.0 Hz), 91.9 (d, J=9.0 Hz), 120.2 (d, J=6.8 Hz), 120.4 (d, J=3.8 Hz), 127.8-130.9, 133.4, 133.4, 137.2, 139.4, 139.8, 149.4-149.6 (2C), 164.5, 164.9.

R₂=4-chlorobenzoyl, β-anomer: ¹H NMR (CDCl₃) δ (ppm): 1.18 (d, 3H, J=6.0 Hz), 2.21 (s, 3H), 2.22 (s, 3H), 3.84-4.00 (m, 2H), 4.49 (d, 1H, J=12.0 Hz), 4.66 (d, 1H, J=12.0 Hz), 5.28-5.38 (m, 2H), 5.49 (s, 1H), 6.97-7.88 (m, 21H). ¹³C NMR (CDCl₃) δ (ppm): 16.1, 20.7 (2C), 69.9, 71.4, 72.4, 73.1, 74.6, 76.3 (d, J=4.5 Hz), 97.4 (d, J=12.8 Hz), 120.2 (d, J=7.5 Hz), 120.7 (d, J=6.0 Hz), 127.6-131.2, 133.5, 133.6, 137.5, 139.6, 139.9, 164.5, 165.0.

R₂=benzoyl, α-anomer: ¹H NMR (CDCl₃) δ (ppm): 1.10 (d, 3H, J=6.0 Hz), 2.31 (s, 6H), 4.19 (dd, 1H, J=3.0 12.0 Hz), 4.43-4.49 (m, 1H), 4.63 (d, 1H, J=12.0 Hz), 4.67 (d, 1H, J=12.0 Hz), 5.67 (dd, 1H, J=1.2 3.3 Hz), 5.79 (dd, 1H, J=3.3 10.5 Hz), 6.10 (dd, 1H, J=3.6 8.4 Hz), 7.08-7.98 (m, 23H). ¹³C NMR (CDCl₃) δ (ppm): 15.9, 20.7 (2C), 66.4, 70.4, 72.0, 72.6, 73.0 (d, J=3.8 Hz), 92.2 (d, J=9.0 Hz), 120.3 (d, J=7.5 Hz), 120.4 (d, J=6.8 Hz), 127.7-128.5, 129.5-130.0, 133.0, 133.2, 133.4, 133.4, 137.4, 149.5, 149.6, 165.8, 165.9.

R₂=benzoyl, β-anomer: ¹H NMR (CDCl₃) δ (ppm): 1.17 (d, 3H, J=6.0 Hz), 2.18 (s, 6H), 3.89-3.97 (m, 2H), 4.51 (d, 1H, J=12.0 Hz), 4.65 (d, 1H, J=12.0 Hz), 5.32-5.37 (m, 2H), 5.51 (dd, 1H, J=0.9 3.6 Hz), 6.95-7.96 (m, 23H). ¹³C NMR (CDCl₃) δ (ppm): 16.1, 20.4 (2C), 70.1, 71.3, 73.2, 74.7, 76.6, 97.4 (d, J=12.8 Hz), 120.2 (d, J=6.8 Hz), 120.8 (d, J=6.0 Hz), 127.5-128.5, 129.2-130.1, 133.2, 133.4, 133.5, 133.6, 137.5, 149.2 (d, J=3.2 Hz), 149.5 (d, J=5.3 Hz), 165.8, 166.1.

### Example 5

### Preparation of Acceptors

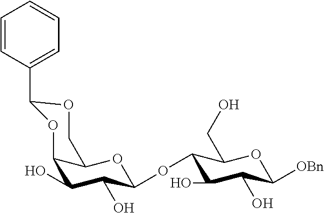

To a suspension of β-benzyllactoside (700 g, 1.6 mol) in DMF (5 L) was added benzaldehyde dimethylacetal (389.5 mL, 2.6 mmol, 1.6 eq.) and p-TsOH.H₂O (31.5 g, 0.17 mmol, 0.1 eq.). The reaction mixture was then heated to 40-44° C. for approximately 22 h, after which a white suspension was obtained. It was cooled to ice bath temperature, ⁱPr₂O (4 L) was added and the resulting suspension was further stirred for 1½ h at this temperature. This mixture was filtrated and the white solid obtained was washed/suspended with ⁱPr₂O (2×1 L). After drying 702 g were obtained of the wanted product as a white free solid. The resulting mother liquor was allowed to stand at room temperature for approximately 3 days, during which time more white solid appeared. This was filtrated and washed/suspended with ⁱPr₂O (2×150 mL). Obtained further 25 g of product. Combined amount: 727 g (86%). ¹H NMR (CD₃OD) δ in ppm: 3.3-3.45 (m, 2H); 3.5-3.7 (m, 5H); 3.95 (m, 2H); 4.15 (m, 3H); 4.40 (d, 1H, J=7.8 Hz); 4.5 (d, 1H, J=7.5 Hz); 4.65 (d, 1H, J=11.8 Hz); 4.9 (d, 1H, J=11.8 Hz); 5.55 (s, 1H); 7.25 (m, 5H). ¹³C NMR: 61.75, 68.33, 70.17, 71.78, 71.86, 73.52, 74.89, 76.33, 77.35, 80.02; 102.3, 103.22, 104.87; 128.8-139.03.

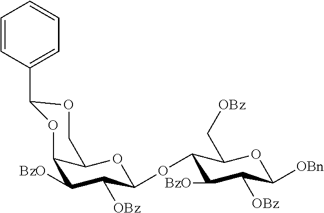

To an ice bath cooled solution of benzylidene-β-benzyllactoside (example 9, 6.2 g, 11.9 mmol) in pyridine (40 mL), BzCl (13.8 mL) was added dropwise. The reaction mixture stirred 30 min at this temperature, and overnight at RT. The reaction was quenched with MeOH and the solvent was evaporated in vacuo. The residue was dissolved in DCM and washed with water, 1N HCl, water, NaHCO₃, and brine. The organic phase was dried over Na₂SO₄ and the solvent was removed in vacuo. The solid obtained was recrystallized form EtOAc/Hexane to yield 9.1 g (73%) of a white pure solid. Mp.: 162-164° C. ¹H NMR (CDCl₃) δ (ppm): 2.95 (m, 1H); 3.58 (m, 1H); 3.78 (m, 2H); 4.25 (m, 2H); 4.40 (dd, 1H, J=4.3 Hz, J=12.1 Hz); 4.55 (d, 1H, 12.5 Hz); 4.65 (m, 1H); 4.70 (d, 1H, J=7.7 Hz); 4.78 (d, 1H, 12.5 Hz); 4.85 (d, 1H, J=7.9 Hz); 5.15 (dd, 1H, J=3.4 Hz, J=10.4 Hz); 5.30 (s, 1H); 5.40 (dd, 1H, J=7.8 Hz, J=9.2 Hz); 5.75 (m, 2H). 7.05-8.05 (m, 35H).

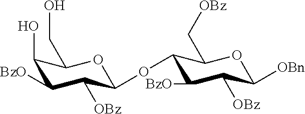

To a solution of the pentabenzoyl derivative (example 14, 22.0 g, 21 mmol) in DCM (140 mL) MeOH (20 mL) mixture TsOH monohydrate (1.6 g, 0.4 eq) was added, and the reaction mixture stirred for 2 days at 40° C. After this time a saturated solution of NaHCO₃ was added and the mixture stirred for 15 min. The phases were separated and the organic one was washed with water and brine; after drying over Na₂SO₄. The solvent was evaporated in vacuo and the solid obtained was suspended in EtOAc (200 mL) and the slurry stirred overnight, after filtration and drying 14.5 g (74%) were obtained. Mp.: 220-222.5° C. ¹H NMR (CDCl₃): 2.95 (m, 1H); 3.35 (m, 3H); 3.80 (m, 1H); 4.20 (m, 2H); 4.40 (dd, 1H, J=5 Hz, J=11.9 Hz); 4.5-4.9 (m, 5H); 5.07 (dd, 1H; J=3.1 Hz, 10.4 Hz); 5.45 (dd, 1H, J=7.7 Hz, J=9.4 Hz); 5.70 (m, 2H), 7.0-8.1 (m, 30H).

Analogously prepared:

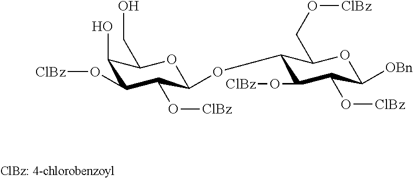

¹H NMR (CDCl₃): 2.95 (m, 1H); 3.40 (m, 3H); 3.85 (m, 1H); 4.15 (m, 2H); 4.40 (dd, 1H, J=5 Hz, J=11.9 Hz); 4.5-4.8 (m, 5H); 5.15 (dd, 1H; J=3.1 Hz, 10.4 Hz); 5.40 (dd, 1H, J=7.7 Hz, J=9.4 Hz); 5.65 (m, 2H), 7.0-8.0 (m, 25H).

Analogously prepared:

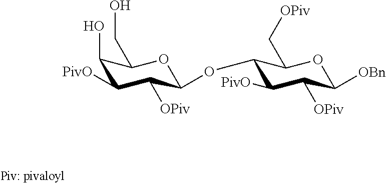

¹H NMR 6 (CD₃OD) in ppm: 1.05-1.2 (4s, 36H); 1.25 (s, 9H); 3.4 (m, 2H); 3.8 (m, 1H); 3.95 (m, 2H); 4.1 (m, 1H); 4.2 (dd, 1H, J=11.8 Hz, J=5.5 Hz); 4.55 (m, 4H); 4.8 (d, 1H, J=12.5 Hz); 4.9 (dd, 1H); 5.0 (dd, 1H); 5.2 (m, 2H); 7.2-7.4 (m, 5H).

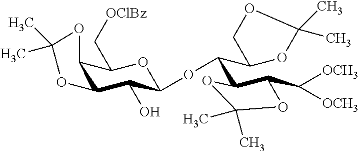

4-O-(6-O-(4-chlorobenzoyl)-3,4-O-isopropylidene-β-D-galactopyranosyl)-2,3:5,6-di-β-isopropylidene-D-glucose dimethyl acetal was prepared according to WO 2010/115935.

### Example 5

### Glycosylation

### Sialylation

To a −20° C. cooled suspension of the donor (1.6 eq) and acceptor (1.0 eq) in DCM/THF triflic acid (0.16 eq) was added. The reaction mixture stirred for 3 h at −20° C. A solution of aqueous NaHCO₃ (was added to neutralize the acid. The phases were separated and the organic phase was washed with water and with water/brine mixture. The organic solvent was evaporated giving rise to the product as syrup.

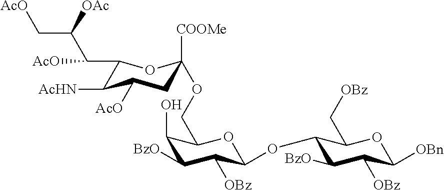

¹H NMR (CDCl₃) δ (ppm): 1.7 (s, 3H); 1.85 (s, 3H); 2.05 (m, 7H); 2.15 (s, 3H); 2.35 (dd, 1H, J=4.7 Hz, J=12.6 Hz); 3.07 (m, 1H); 3.3 (m, 1H); 3.4 (m, 1H); 3.7-3.85 (m, 4H); 3.9-4.1 (m, 4H); 4.17 (m, 1H); 4.4-4.6 (m, 3H); 4.6-4.9 (m, 4H); 5.05-5.35 (m, 4H); 5.45 (dd, 1H; J=7.8 Hz, J=9.6 Hz); 5.6-5.75 (m, 2H); 7.0-7.7 (m, 20H); 7.8-8.1 (m, 10H). ¹³C NMR (CDCl₃) δ (ppm): 20.55, 21.07, 21.12, 21.16, 21.24, 23.26, 35.73, 49.40, 53.26, 54.67, 60.24, 62.87, 62.97, 65.57, 67.88, 69.32, 70.15, 70.29, 70.56, 72.12, 72.86, 73.29, 73.59, 74.18, 74.51, 98.90, 99.02, 101.68, 127.88, 127.95, 128.05, 128.51, 128.59, 128.64, 129.15, 129.53, 129.60, 128.88, 130.07, 130.16, 133.33, 133.40, 133.69, 136.71, 165.22, 164.42, 165.50, 166.01, 166.07, 167.85, 170.47, 170.56, 170.65, 171.14, 171.39.

The same product was obtained using donors having R^{a}═H, methoxy and bromo.

Analogously prepared:

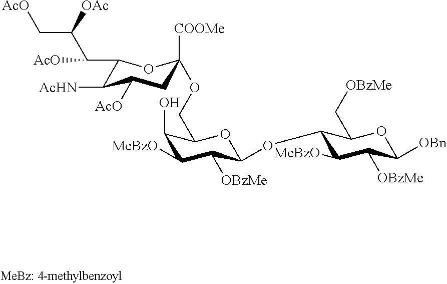

¹H NMR (CDCl₃) δ (ppm): 1.85 (s, 3H); 1.95 (m, 1H); 2.05, 2.07, 2.11, 2.15 (4s, 12H); 2.3 (s, 3H); 2.35-2.45 (m, 13H); 3.0 (d, 1H, J=5.8 Hz); 3.25 (m, 1H); 3.32 (m, 1H); 3.45 (m, 1H); 3.75-3.85 (m, 4H); 3.95-4.05 (m, 3H); 4.1 (m, 1H); 4.25 (dd, 1H, J=9.15 Hz, J=9.55 Hz); 4.37 (dd, 1H, J=12 Hz, J=2.0 Hz); 4.42 (dd, 1H, J=5.1 Hz, J=12 Hz); 4.53 (d, 1H; J=12.5 Hz); 4.62 (dd, 1H; J=1.4 Hz, J=11.6 Hz); 4.67 (d, 1H, J=7.7 Hz); 4.76 (d, 1H, 12.6 Hz); 4.79-4.89 (m, 2H); 5.1 (dd, 1H, J=3 Hz, J=12.4 Hz); 5.2-5.35 (m, 3H); 5.37 (dd. 1H, J=7.8 Hz, J=9.3 Hz) 5.6-5.75 (m, 2H); 7.1-7.3 (m, 15H); 7.7-7.9 (m, 10H). ¹³C NMR (CDCl₃) δ (ppm): 20.75, 21.05, 21.14, 21.27, 21.66, 21.71, 21.85, 21.96, 23.42, 34.61, 36.39, 45.98, 49.59, 53.23, 54.66, 59.01, 60.73, 62.85, 65.85, 67.74, 69.14, 69.79, 69.96, 70.39, 72.16, 72.81, 73.19, 73.22, 73.74, 74.22, 76.71, 98.96 (2C), 101.58, 125.53, 126.83, 127.25, 127.90, 127.96, 128.46, 128.49, 129.15, 129.21, 129.26, 129.36, 129.88, 130.05, 130.12, 130.17, 136.83, 138.10, 143.72, 143.86, 143.96, 144.02, 144.06, 165.29, 165.54, 166.08, 167.84, 170.42, 170.48, 170.52, 171.18, 171.22.

Analogously prepared:

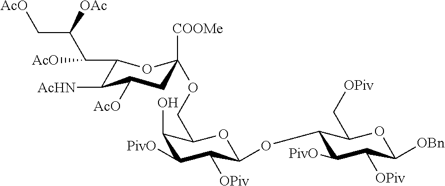

¹H NMR (CDCl₃) δ in ppm: 1.05-1.3 (m, 45H); 1.75-2.25 (m, 16H); 2.6 (dd, 1H, J=4.7 Hz, 12.6 Hz); 3.5-3.75 (m, 8H); 3.9-4.10 (m, 5H), 4.3 (m, 2H); 4.5 (m, 4H); 4.75-5 (m, 4H); 5.2 (m, 3H); 5.3 (m, 2H); 7.25 (m, 5H). ¹³C NMR (CDCl₃) δ (ppm): 21.02; 21.1; 21.32; 23.09; 23.45; 27.33; 27.38; 27.43; 27.51; 38.90; 39.08; 53.24; 62.34; 62.68; 62.70; 62.72; 66.44; 67.46; 68.87; 69.09; 69.4; 70.56; 71.78; 71.83; 73.00; 73.13; 73.43; 73.54; 73.88; 73.89; 99.26; 99.38; 99.92; 128.17; 128.57; 136.84; 168.09; 170.38; 170.41; 171.00; 171.22; 178.00.

Analogously prepared:

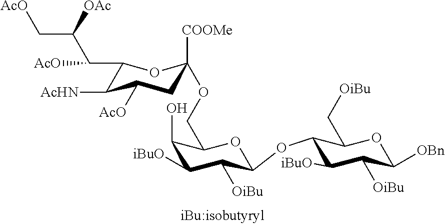

¹H NMR (CDCl₃) δ in ppm: 1.0-1.25 (m, 30H); 1.8-2.2 (m, 16H); 2.4-2.65 (m, 6H); 3.5-3.9 (m, 9H); 3.95-4.1 (m, 3H); 4.13-4.25 (m, 2H); 4.4-4.6 (m, 5H); 4.78-4.9 (m, 3H); 4.97 (dd, 1H, J=7.95 Hz, J=9.75 Hz); 5.13-5.25 (m, 3H); 5.3 (m, 2H); 7.2-7.35 (m, 5H). ¹³C NMR (CDCl₃) δ (ppm): 18.97, 19.08, 19.13, 19.21, 19.34, 19.58, 21.09, 21.30, 23.43, 34.13, 49.56, 50.29, 54.66, 62.09, 62.66, 66.37, 67.40, 68.87, 69.48, 70.69, 71.42, 71.96, 72.87, 73.02, 73.49, 75.35, 99.23, 99.35, 100.60, 127.99, 128.14, 128.58, 136.92, 168.05, 170.24, 170.37, 170.41, 171.04, 175.26, 175.71, 176.11, 176.29, 176.71.

### Example 6

### Glycosylation

### Fucosylation

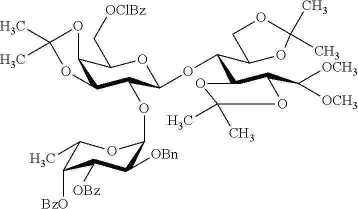

To a cooled solution of 2-O-benzyl-3,4-di-O-benzoyl-α-L-fucopyranosyl di-(4-methylphenyl) phosphite (1.2 eq) and 4-O-(6-O-(4-chlorobenzoyl)-3,4-O-isopropylidene-β-D-galactopyranosyl)-2,3:5,6-di-O-isopropylidene-D-glucose dimethyl acetal (1.0 eq) in DCM/2-methyltetrahydrofurane, triflic acid (0.16 eq.) was added. The reaction mixture stirred for 1 h at 0° C. and then a solution of aqueous NaHCO₃ was added to neutralize the acid. The phases were separated and the organic phase was washed with water and with water/brine mixture. The organic solvent was evaporated giving rise to the product as syrup. Characterization data were in accordance with those published in WO 2010/115935.

### Example 7

### Deprotection

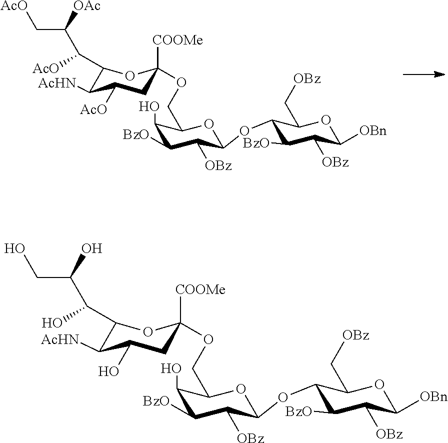

The syrup obtained above was diluted with MeOH, cooled to 5° C. and sulfuric acid was added dropwise. The reaction mixture was stirred for 48 h at this temperature and neutralized with Et₃N. MeOH was evaporated and the residue was dissolved in EtOAc, washed with water once and with 5/1 water brine. The organic phase was evaporated in vacuo and the residue was crystallized (yield: 50-60% for two steps). ¹H NMR (CD₃OD) δ (ppm): 1.5 (dd, 1H, J=11.7 Hz, J=12.6 Hz); 2.0 (s, 3H); 2.45 (dd, 1H, J=4.6 Hz, J=12.8 Hz); 3.2 (m, 1H); 3.4-3.7 (m, 7H); 3.7-3.85 (m, 5H); 3.95 (m, 1H); 4.1 (d, 1H, J=3.5 Hz); 4.25 (dd, 1H, J=9.2 Hz, J=9.5 Hz); 4.45-4.65 (m, 3H); 4.75 (d, 1H, J=12.2 Hz); 5.15 (dd, 1H, J=3.3 Hz, J=10.4 Hz); 5.35 (dd, 1H, J=8 Hz, J=9.7 Hz); 5.64 (dd, 1H, J=7.9 Hz, J=10.3 Hz); 5.69 (dd, 1H, J=9.2 Hz, J=9.4 Hz); 7.05-7.65 (m, 20H, 7.8-8.1 (m, 10H). ¹³C NMR (CD₃OD) δ (ppm): 21.62, 39.03, 52.37, 52.44, 60.04, 63.14, 65.25, 67.44, 68.41, 70.50, 70.87, 72.58, 73.24, 73.39, 73.66, 73.97, 74.79, 77.15, 127.73, 128.16, 128.27, 128.37, 128.63, 129.08, 129.41, 129.46, 129.49, 129.54, 129.59, 129.72, 129.81, 129.89, 133.18, 137.18, 165.63, 165.66, 165.95, 166.01, 166.22, 169.35, 173.99.

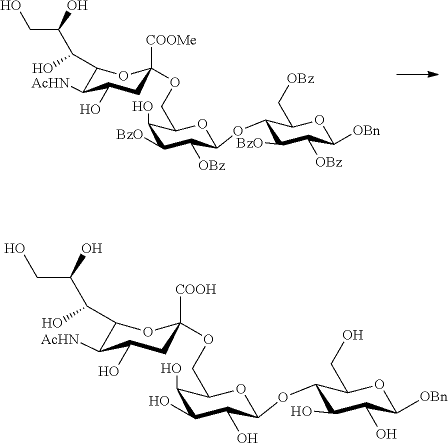

To a solution of the partially protected trisaccharide obtained above in 5 volumes of MeOH 1 M aqueous solution of NaOH was added slowly. The reaction mixture stirred overnight at RT and Amberlite IR 120 H⁺ was added to neutralize the base. The mixture was filtered and the solvent was evaporated in vacuo. The material was then dissolved in MeOH and EtOH, and TBME was added slowly. The precipitate was filtered off and washed twice with TBME. The white solid obtained was dissolved in 1 M NaOH and the reaction mixture was stirred for 6 h. IR-120 H⁺ was used to neutralize the base and the solvent was evaporated in vacuo. After coevaporation with EtOH (twice) the product was dissolved in MeOH and iPrOH was added slowly to give a solid in free acid form. ¹H NMR (CD₃OD) δ (ppm): 1.63 (t, 1H, J=11.9 Hz); 2.00 (s, 3H); 2.78 (dd, 1H, J=4.5 Hz, J=12.2 Hz); 3.28-3.49 (m, 4H); 3.50-3.79 (m, 9H); 3.80-3.97 (m, 5H); 4.02 (dd, 1H, J=7.5 Hz, J=10 Hz); 4.35 (m, 1H); 4.42 (d, 1H, J=7.8 Hz); 4.66 (d, 1H, J=11.7 Hz); 7.22-7.37 (m, 3H); 7.38-7.46 (m, 2H). ¹³C NMR (CD₃OD) δ (ppm): 21.55, 41.26, 52.70, 60.81, 63.21, 63.42, 68.56, 69.01, 69.30, 70.66, 71.15, 72.12, 73.08, 73.55, 73.78, 74.47, 75.28, 75.32, 80.03, 100.29, 101.89, 103.36, 127.36, 127.87, 128.01, 128.12, 137.72, 173.36, 173.88.

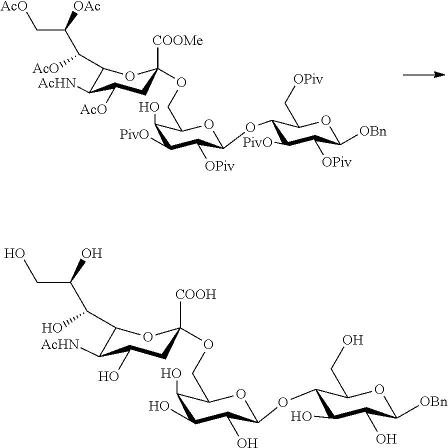

To a solution of a protected trisaccharide in MeOH NaOMe was added and the reaction mixture was stirred for 5 h at 45° C. The cooled solution was washed with heptane and acetone was added. The precipitate was filtered off and dissolved in 1 M NaOH and the reaction mixture was stirred for 6 h. IR-120 H⁺ was used to neutralize the base and the solvent was evaporated in vacuo. After coevaporation with EtOH (twice) the product was dissolved in MeOH and iPrOH was added slowly to give a solid in free acid form.

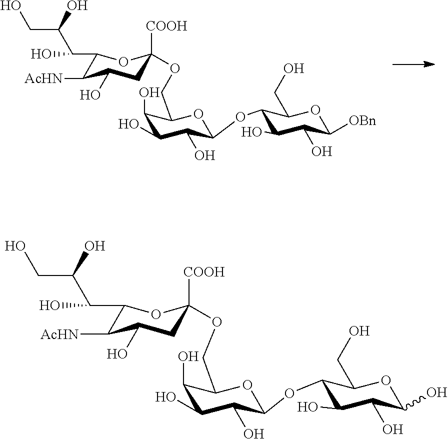

To a solution of 40 g of free acid in a mixture of methanol and water (250 mL+300 mL) 4 g of Pd/C (10%) were added. The reaction mixture was stirred 2 d at RT under H₂ pressure (balloon). The mixture was then filtered through a pad of Celite and the solvent was evaporated in vacuo. The residue was dissolved in 80 mL of H₂O and dropped to 1200 mL of EtOH. The slurry was filtrated, the solid was washed with EtOH, acetone and a mixture of 1/1 acetone/Et₂O. The solid was dried to give 35 g of 6′-sialyllactose. ¹H NMR (D₂O) (anomeric mixture of glucose 0.6/0.4 β/α): 1.75 (dd, 1H, J=12.0 Hz, J=11.9 Hz); 2.05 (s, 3H); 2.7 (dd, 1H, J=12.0 Hz, J=4.6 Hz); 3.31 (dd, 0.6H, J=7.8 Hz, J=8.9 Hz); 3.5-3.75 (m, 11.4H), 3.76-4.05 (m, 8.9H); 4.43 (d, 1H, J=7.8 Hz); 4.67 (d, 0.6H, J=7.8 Hz), 5.23 (d, 0.4H, J=3.8 Hz). ¹³C NMR: 19.51, 24.78, 42.82, 54.51, 60.15, 62.83, 62.99, 65.36, 66.3, 71.1, 71.24, 72.68, 73.51, 73.78, 74.35, 74.53, 75.09, 75.24, 76.42, 76.45, 77.35, 77.39, 82.35, 82.46, 94.54, 98.37, 103.01, 105.92, 105.95, 176.21, 177.64.

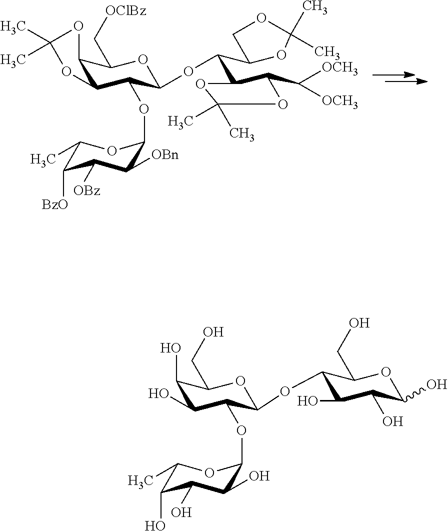

For deprotection of O-(2-O-benzyl-3,4-di-O-benzoyl-α-L-fucopyranosyl)-(1→2)—O-(6-O-(4-chlorobenzoyl)-3,4-isopropylidene-β-D-galactopyranosyl)-(1→4)-2,3:5,6-di-O-isopropylidene-D-glucose dimethyl acetal to 2′-fucosyllactose see WO 2010/115935.

Having thus described the present invention in detail and the advantages thereof, it is to be understood that the detailed description is not intended to limit the scope of the invention thereof.

1.-28. (canceled)

29. A method for the manufacture of a mixture of human milk oligosaccharides (HMOs), comprising a step of subjecting a mixture of at least two compounds selected from the group consisting of compounds of general formulae 1 and 2 to catalytic hydrogenolysis 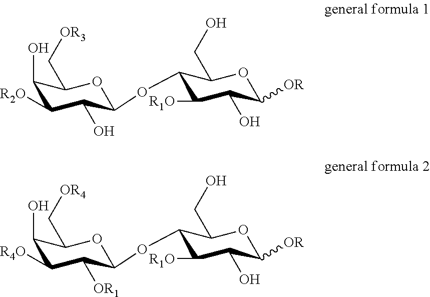
wherein
R is a group removable by catalytic hydrogenolysis,
R₁ is independently fucosyl or H,
R₂ is selected from N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₃ is H or N-acetyl-lactosaminyl group optionally substituted with a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₄ is independently sialyl or H,
and salts thereof,
with the proviso that at least one of R₁ or R₄ is not H in general formula 2.

30. The method according to claim 29, wherein the compounds are selected from the group of: R-glycosides of 2′-fucosyllactose, 3-fucosyllactose, 2′,3-difucosyllactose, 3′-sialyllactose, 6′-sialyllactose, 3′-sialyl-3-fucosyllactose, lacto-N-tetraose, and lacto-N-neotetraose.

31. The method according to claim 29, wherein the catalytic hydrogenolysis is carried out in at least one protic solvent, preferably in water or in aqueous alcohol, in the presence of a hydrogenolysis catalyst, preferably Pd on charcoal or Pd black.

32. The method according to claim 29, wherein the compounds of general formulae 1 are selected from the group consisting of compounds of general formulae 1a, 1b and wherein the compounds of general formula 2 are selected from the group consisting of compounds of general formula 2 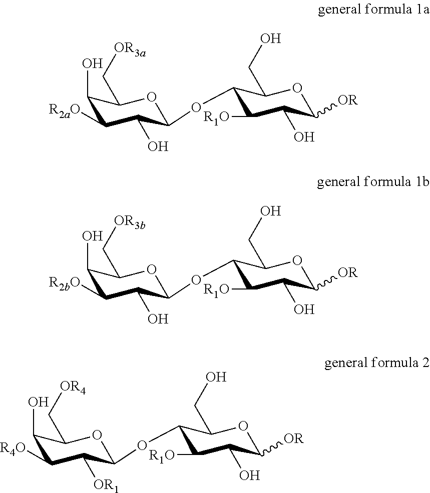
wherein R, R₁ and R₄ are as defined in claim 29,
R₂ₐ is N-acetyl-lactosaminyl group optionally substituted with a glycosyl residue comprising one N-acetyl-lactosaminyl and/or one lacto-N-biosyl group; any N-acetyl-lactosaminyl and lacto-N-biosyl group can be substituted with one or more sialyl and/or fucosyl residue,
R₃ₐ is H or N-acetyl-lactosaminyl group optionally substituted with a lacto-N-biosyl group; any N-acetyl-lactosaminyl and lacto-N-biosyl group can be substituted with one or more sialyl and/or fucosyl residue,
R_{2b} is lacto-N-biosyl group optionally substituted with sialyl and/or fucosyl residue,
R_{3b} is H or N-acetyl-lactosaminyl group optionally substituted with one or two N-acetyl-lactosaminyl and/or one lacto-N-biosyl group; any N-acetyl-lactosaminyl and lacto-N-biosyl group can be substituted with one or more sialyl and/or fucosyl residue,
and salts thereof.

33. The method according to claim 32, wherein
the N-acetyl-lactosaminyl group in the glycosyl residue of R₂ₐ in general formula 1a is attached to the another N-acetyl-lactosaminyl group with 1-3 interglycosidic linkage,
the lacto-N-biosyl group in the glycosyl residue of R₂ₐ in general formula 1a is attached to the N-acetyl-lactosaminyl group with 1-3 interglycosidic linkage,
the lacto-N-biosyl group in the glycosyl residue of R₂ₐ in general formula 1a is attached to the N-acetyl-lactosaminyl group with 1-3 interglycosidic linkage,
the N-acetyl-lactosaminyl group in the glycosyl residue of R_{3b} in general formula 1b is attached to the another N-acetyl-lactosaminyl group with 1-3 or 1-6 interglycosidic linkage,
the lacto-N-biosyl group in the glycosyl residue of R_{3b} in general formula 1b is attached to the N-acetyl-lactosaminyl group with 1-3 interglycosidic linkage.

34. The method according to claim 33, wherein general formula 1a represents the R-glycosides of lacto-N-neotetraose, para-lacto-N-hexaose, para-lacto-N-neohexaose, lacto-N-neohexaose, para-lacto-N-octaose and lacto-N-neooctaose optionally substituted with one or more sialyl and/or fucosyl residue, and general formula 1b represents the R-glycosides of lacto-N-tetraose, lacto-N-hexaose, lacto-N-octaose, iso-lacto-N-octaose, lacto-N-decaose and lacto-N-neodecaose optionally substituted with one or more sialyl and/or fucosyl residue.

35. The method according to claim 29, wherein
the fucosyl residue attached to the N-acetyl-lactosaminyl and/or the lacto-N-biosyl group is linked to
the galactose of the lacto-N-biosyl group with 1-2 interglycosidic linkage and/or
the N-acetyl-glucosamine of the lacto-N-biosyl group with 1-4 interglycosidic linkage and/or
the N-acetyl-glucosamine of the N-acetyl-lactosaminyl group with 1-3 interglycosidic linkage,
the sialyl residue attached to the N-acetyl-lactosaminyl and/or the lacto-N-biosyl group is linked to
the galactose of the lacto-N-biosyl group with 2-3 interglycosidic linkage and/or
the N-acetyl-glucosamine of the lacto-N-biosyl group with 2-6 interglycosidic linkage and/or
the galactose of the N-acetyl-lactosaminyl group with 2-6 interglycosidic linkage.

36. The method according to claim 29, wherein the compounds are selected from the group of: R-glycosides of 2′-fucosyllactose, 3-fucosyllactose, 2′,3-difucosyllactose, 3′-sialyllactose, 6′-sialyllactose, 3′-sialyl-3-fucosyllactose, lacto-N-tetraose, lacto-N-neotetraose, LNFP-I, LNFP-II, LNFP-III, LNFP-V, LST-a, LST-b, LST-c, FLST-a, FLST-b, FLST-c, LNDFH-I, LNDFH-II, LNDFH-III, DS-LNT, FDS-LNT I and FDS-LNT II, and salts thereof.

37. The method according to claim 29, wherein the R-glycoside is β-anomer, and salts thereof.

38. The method according to claim 29, wherein R is benzyl, and salts thereof.

39. The method according to claim 29, wherein the catalytic hydrogenolysis leads to human milk oligosaccharides (HMOs) according to general formulae 3 and 4 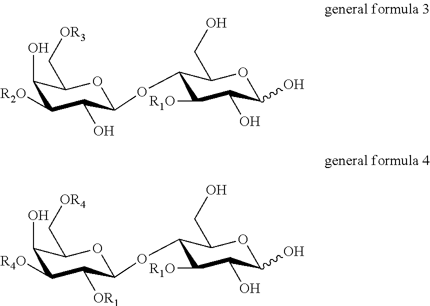
wherein R₁ is independently fucosyl or H,
R₂ is selected from N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₃ is H or N-acetyl-lactosaminyl group optionally substituted with a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₄ is independently sialyl or H,
and salts thereof,
with the proviso that at least one of R₁ or R₄ is not H in general formula 4.

40. The method according to claim 39, wherein the HMOs are characterized by general formulae 3a, 3b and 4 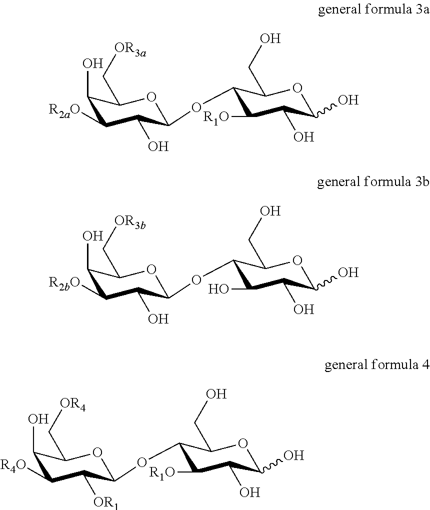
wherein R₁ and R₄ are as defined in claim 39,
R₂ₐ is N-acetyl-lactosaminyl group optionally substituted with a glycosyl residue comprising one N-acetyl-lactosaminyl and/or one lacto-N-biosyl group; any N-acetyl-lactosaminyl and lacto-N-biosyl group can be substituted with one or more sialyl and/or fucosyl residue,
R₃ₐ is H or N-acetyl-lactosaminyl group optionally substituted with a lacto-N-biosyl group; any N-acetyl-lactosaminyl and lacto-N-biosyl group can be substituted with one or more sialyl and/or fucosyl residue,
R_{2b} is lacto-N-biosyl group optionally substituted with sialyl and/or fucosyl residue,
R_{3b} is H or N-acetyl-lactosaminyl group optionally substituted with one or two N-acetyl-lactosaminyl and/or one lacto-N-biosyl group; any N-acetyl-lactosaminyl and lacto-N-biosyl group can be substituted with one or more sialyl and/or fucosyl residue,
and salts thereof.

41. The method according to claim 40, wherein
the N-acetyl-lactosaminyl group in the glycosyl residue of R₂ₐ in general formula 3a is attached to the another N-acetyl-lactosaminyl group with 1-3 interglycosidic linkage,
the lacto-N-biosyl group in the glycosyl residue of R₂ₐ in general formula 3a is attached to the N-acetyl-lactosaminyl group with 1-3 interglycosidic linkage,
the lacto-N-biosyl group in the glycosyl residue of R₃ₐ in general formula 3a is attached to the N-acetyl-lactosaminyl group with 1-3 interglycosidic linkage,
the N-acetyl-lactosaminyl group in the glycosyl residue of R_{3b} in general formula 3b is attached to the another N-acetyl-lactosaminyl group with 1-3 or 1-6 interglycosidic linkage,
the lacto-N-biosyl group in the glycosyl residue of R_{3b} in general formula 3b is attached to the N-acetyl-lactosaminyl group with 1-3 interglycosidic linkage.

42. The method according to claim 41, wherein general formula 3a represents lacto-N-neotetraose, para-lacto-N-hexaose, para-lacto-N-neohexaose, lacto-N-neohexaose, para-lacto-N-octaose and lacto-N-neooctaose optionally substituted with one or more sialyl and/or fucosyl residue, and general formula 3b represents lacto-N-tetraose, lacto-N-hexaose, lacto-N-octaose, iso-lacto-N-octaose, lacto-N-decaose and lacto-N-neodecaose optionally substituted with one or more sialyl and/or fucosyl residue.

43. The method according to claim 41, wherein
the fucosyl residue attached to the N-acetyl-lactosaminyl and/or the lacto-N-biosyl group is linked to
the galactose of the lacto-N-biosyl group with 1-2 interglycosidic linkage and/or
the N-acetyl-glucosamine of the lacto-N-biosyl group with 1-4 interglycosidic linkage and/or
the N-acetyl-glucosamine of the N-acetyl-lactosaminyl group with 1-3 interglycosidic linkage,
the sialyl residue attached to the N-acetyl-lactosaminyl and/or the lacto-N-biosyl group is linked to
the galactose of the lacto-N-biosyl group with 2-3 interglycosidic linkage and/or
the N-acetyl-glucosamine of the lacto-N-biosyl group with 2-6 interglycosidic linkage and/or
the galactose of the N-acetyl-lactosaminyl group with 2-6 interglycosidic linkage.

44. The method according to claim 39, wherein the HMOs are selected from the group of: 2′-fucosyllactose, 3-fucosyllactose, 2′,3-difucosyllactose, 3′-sialyllactose, 6′-sialyllactose, 3′-sialyl-3-fucosyllactose, lacto-N-tetraose, lacto-N-neotetraose, LNFP-I, LNFP-II, LNFP-III, LNFP-V, LST-a, LST-b, LST-c, FLST-a, FLST-b, FLST-c, LNDFH-I, LNDFH-II, LNDFH-III, DS-LNT, FDS-LNT I and FDS-LNT II, and salts thereof.

45. The method according to claim 29, wherein the catalytic hydrogenolysis of the mixture of individual compounds characterized by general formulae 1 and 2 comprising 0-100% of compounds containing one or more sialyl residue but devoid of fucosyl residue, 0-100% of compounds containing one or more fucosyl residue but devoid of sialyl residue, 0-100% of compounds containing one or more sialyl and one or more fucosyl residue and 0-100% of compounds devoid of sialyl and fucosyl residue leads to a mixture of individual compounds characterized by general formulae 3 and 4 comprising 0-100% of compounds containing one or more sialyl residue but devoid of fucosyl residue, 0-100% of compounds containing one or more fucosyl residue but devoid of sialyl residue, 0-100% of compounds containing one or more sialyl and one or more fucosyl residue and 0-100% of compounds devoid of sialyl and fucosyl residue.

46. The method according to claim 29, wherein the method further comprises preparation of the mixture or blend of compounds of general formulae 1 and 2 by the following steps:
a) providing at least one fucosyl, sialyl, N-acetyllactosaminyl or lacto-N-biosyl donor,
b) providing at least one acceptor selected from lactose R-glycoside, LNT R-glycoside and LNnT R-glycoside, wherein R is as defined in claim 29,
c) preparing a blend from compounds provided by steps a) and b);
d) adding at least one enzyme comprising a transglycosidase activity and/or a glycosynthase activity to the blend of step c) thereby forming a mixture;
e) incubating the mixture obtained according to step d); and
f) optionally repeating any of steps a) to d), preferably with the mixture obtained according to step e).

47. The method according to claim 29, wherein the method further comprises preparation of the mixture or blend of compounds of general formulae 1 and 2 by the following steps:
a) providing at least one compound or a mixture of compounds selected from:
optionally sialylated and/or fucosylated lactose derivative of general formula 2: 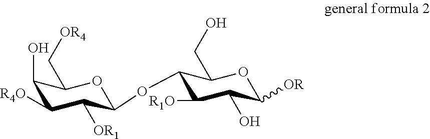
wherein
R is a group removable by hydrogenolysis,
R₁ independently of each other is fucosyl or H
R₄ independently of each other is sialyl or H,
or salts thereof,
preferably provided that the compound of general formula 2 is not R-glycoside of lactose, if provided alone;
a lacto-N-tetraose (LNT) derivative of following formula: 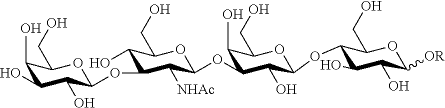
wherein R is a group removable by hydrogenolysis; and
a lacto-N-neotetraose (LNnT) derivative of following formula: 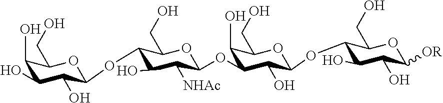
wherein R is a group removable by hydrogenolysis;
b) adding at least one enzyme comprising a transglycosidase activity to the at least one compound or a mixture of compounds provided according to step a);
c) incubating the mixture obtained according to step b); and
d) optionally repeating any of steps a) to c), preferably with the mixture obtained according to step c).

48. A composition comprising a mixture of at least two compounds selected from compounds of general formulae 1 and 2 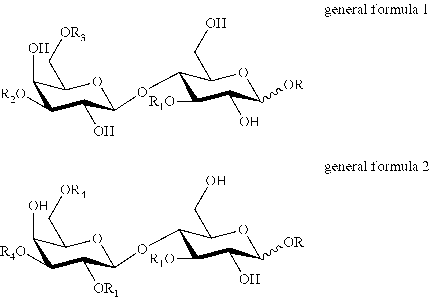
wherein R is a group removable by catalytic hydrogenolysis,
R₁ is independently fucosyl or H,
R₂ is selected from N-acetyl-lactosaminyl and lacto-N-biosyl groups, wherein the N-acetyl lactosaminyl group may carry a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₃ is H or N-acetyl-lactosaminyl group optionally substituted with a glycosyl residue comprising one or more N-acetyl-lactosaminyl and/or one or more lacto-N-biosyl groups; any N-acetyl-lactosaminyl and lacto-N-biosyl group can be substituted with one or more sialyl and/or fucosyl residue,
R₄ is independently sialyl or H,
and salts thereof,
provided that at least one of R₁ or R₄ is not H in general formula 2.
